(19) United States
(12) Patent Application Publication
(10) Pub. No.: US 2013/0183707 A1
(43) Pub. Date: Jul. 18, 2013

(54) STEM CELL BIOINFORMATICS

(71) Applicants: The Charles Stark Draper Laboratory, Inc., Cambridge, MA (US); UNIVERSITY OF PITTSBURGH - OF THE COMMONWEALTH SYSTEM OF HIGHER EDUCATION, Pittsburgh, PA (US)

(72) Inventors: Rami Mangoubi, Newton, MA (US); Paul Sammak, Pittsburgh, PA (US); Mukund Desai, Needham, MA (US); Nathan Lowry, Lexington, MA (US)

(73) Assignees: UNIVERSITY OF PITTSBURGH - OF THE COMMONWEALTH SYSTEM OF HIGHER EDUCATION, Pittsburgh, PA (US); THE CHARLES STARK DRAPER LABORATORY, INC., Cambridge, MA (US)

(21) Appl. No.: 13/741,184

(22) Filed: Jan. 14, 2013

Related U.S. Application Data
(60) Provisional application No. 61/586,651, filed on Jan. 13, 2012.

(51) Int. Cl. C12Q 1/02

(52) U.S. Cl. CPC C12Q 1/02; G06F 19/10
USPC 435/29; 435/288.7; 702/19

(57) ABSTRACT

Ex vivo cell culture, especially the culture of stem cells, is a valuable and widely used technique. The appearance of unlabeled cultured cells contains significant information about the cell's identity, including its differentiation status and lineage. However, mere visual inspection of cells is a subjective process subject to inconsistencies between microscopists. This disclosure provides methods of quantifying cells' appearance, validating identity with known biomarkers, allowing automated classification of cells as well as automated segmentation and delineation of the borders of a cell colony. Also provided are systems and methods for comparing and standardizing cells cultured by different scientists using different cell culture methods.

### CROSS-REFERENCE TO RELATED PATENT APPLICATIONS

This application claims the benefit of and priority to U.S. Application No. 61/586,651, filed Jan. 13, 2012, which is incorporated herein by reference in its entirety.

### GOVERNMENT SUPPORT

This invention was made with government support under Grants RO1 EB006161 and GM077872 awarded by the National Institutes of Health. The government has certain rights in the invention.

### BACKGROUND

Stem cells show immense promise in the fields of drug screening, tissue engineering, and cell therapy. However, current methods for characterizing stem cells are limited by certain disadvantages. Most researchers use visual microscopic inspection to monitor the phenotype of self-renewing or differentiating stem cells without perturbing the cell culture, but for definitive characterization lineage-selective molecular markers are usually applied to subsamples that are sacrificed for that purpose. There is a need for real-time monitoring of the phenotype of live-cells in a non-invasive and non-destructive manner, whether for classifying the structural organization of cells and colonies, differentiation status, or for automatically identifying the boundaries of cells. There is also a need for systems that facilitate collaborations between researchers by quantifying comparisons between different cultures of cells.

### SUMMARY

According to one aspect of the disclosure, a method of identifying borders of a cluster of neighboring cells includes obtaining an image of a cluster of neighboring cells; representing the image as a multiplicity of pixels; segmenting the image using texton analysis, thereby identifying the borders of the cluster of neighboring cells; and identifying segments devoid of cells between at least some cells within the cluster of neighboring cells.

In some implementations, the method does not require a user to manually identify the cluster of neighboring cells. In certain implementations, calculating a texton includes calculating at least eight filter responses at a given pixel, wherein at least one of the filter responses is derived from a Gaussian filter, a Laplacian-of-Gaussian filter, or a bar filter. In some of these implementations, texton analysis includes analyzing a cell texture including performing wavelet decomposition or any multiresolution decomposition algorithm. In yet other implementations, the wavelet decomposition analysis or multiresolution decomposition algorithm is an n-level decomposition that yields three detail subbands per level.

In other implementations, the cluster of neighboring cells is a stem cell colony, a colony of differentiated cells, a colony of trophectoderm cells, or a colony of neuronal cells.

According to another aspect of the disclosure, a method of classifying test cells includes obtaining an image of one or more test cells; representing the image as a multiplicity of pixels; calculating a texton of at least a subset of said multiplicity of pixels. Calculating the texton further includes calculating at least one filter response at a given pixel and using a processor to compare the texton to one or more reference textons using one or more statistical comparison methods. Additionally, the method includes identifying the reference cell that most closely matches the test cell based on the comparison; whereby the test cells are classified as belonging to a class corresponding to the identified reference.

In some implementations, calculating the texton includes calculating at least eight filter responses at a given pixel. In other implementations, using a Gaussian filter, a Laplacian-of-Gaussian filter, or a bar filter cell texture is analyzed. In yet other implementations, analyzing cell texture includes performing wavelet decomposition analysis or any multiresolution decomposition algorithm. In some implementations, the analysis is preformed on cells within a border identified using a segmentation algorithm.

According to yet another aspect of the disclosure, a method of classifying test cells includes obtaining an image of one or more test cells; representing the image as a multiplicity of pixels; analyzing a texture of at least a subset of said multiplicity of pixels; comparing the texture with at least five library textures derived from a library of reference cells. The method also includes applying one or more statistical comparison methods to compare the textures. The library includes cells of at least three differentiation states. Additionally the method includes identifying the reference cell that most closely matches the test cell based on the comparison, whereby the test cells are classified as belonging to a class corresponding to the identified reference cell.

In some implementations, the library also includes at least two different lineages. In other implementations, the reference cell types in the library are selected from at least two of a mouse cell, a human cell, an embryonic stem cell, an induced pluripotent cell, a neural stem cell, a kidney cell, a trophectoderm cell, a neurectoderm cell, a fibroblast, and an oligodendrocyte precursor cell. One or more statistical comparison methods include a comparison of probability density functions, or estimates thereof in some implementations. In certain implementations the one or more statistical comparison methods include a Kullback-Leibler Distances comparison, parametric or non-parametric binary or M-ary hypothesis test. In other implementations, analyzing cell texture includes performing wavelet decomposition analysis or any multiresolution decomposition algorithm, wherein the wavelet decomposition analysis or multiresolution decomposition algorithm is an n-level decomposition that yields three detail subbands per level.

According to another aspect of the disclosure, a method of comparing cells includes providing a database suitable for storing cell culture condition data and cell image data; receiving cell culture condition data and cell image data provided by a first user; calculating a similarity between the cell image data and cell culture condition data provided by the first user to the cell image data and cell culture condition data previously stored in the database using one or more statistical comparison methods; and then transmitting the similarity to at least the first user.

In some implementations, the method also includes receiving cell culture condition data and cell image data provided by a second user; and calculating a second similarity between the cell image data and cell culture condition data provided by the first user to the cell image data and cell culture condition data provided by the second user using one or more statistical comparison methods. In certain implementations, the cell image data comprises a micrograph, textural information derived from a micrograph, or information derived from a micrograph using wavelet decomposition. The micrograph is obtained by phase contrast microscopy, fluorescence microscopy, or electron microscopy phase contrast, fluorescence microscopy, luminescence microscopy, magnetic resonance imaging, ultrasound imaging, or widefield microscopy, confocal microscopy, tomographic reconstruction, or statistical reassignment in other implementations.

In certain implementations, the method also includes displaying the cell culture condition data and cell image data previously stored in the database to the first user. In some of these implementations, the cell culture condition data provided by the first user or the second user includes conditions appropriate for long-term cell maintenance or experimental conditions. The database is accessible via the Internet in certain implementations. In other implementations, the one or more statistical methods include a parametric or non-parametric binary or M-ary hypothesis test. In some implementations, the similarity is calculated using a Probability Density Function estimator and quantified using information divergence, or by applying Kullback-Leibler Distances.

In certain implementations, a processor identifies the similarity between image data of a first cell and image data of a second cell, and predicts non-image data about the first cell based on non-image data about the second cell. In some of these implementations, the non-image data comprises gene expression data, protein level data, small molecule level data, or enzymatic activity data.

According to yet another aspect of the disclosure, a system that compares cells includes a storage device that stores cell culture condition data provided by a first user and cell image data provided by the first user, and cell culture condition data provided by a second user and cell image data provided by the second user. The system also includes a computer application configured to calculate a similarity between the cell image data provided by the first user to the cell image data provided by the second user using one or more statistical comparison methods. The computer program is also configured to transmit the similarity to at least one of the first user and the second user.

In certain implementations, the cell image data includes at least one of a micrograph, obtained by phase contrast microscopy, fluorescence microscopy, or electron microscopy; textual information derived from a micrograph; and information derived from a micrograph using wavelet decomposition.

In other implementations, the cell culture condition data provided by the first user or the second user includes conditions appropriate for long-tem cell maintenance or experimental conditions. The first and second user are connected to the storage device by a network, and, in some implementation, are different users. One or more statistical comparison methods comprises a comparison of probability density functions, or estimates thereof are used in some implementations. The one or more statistical comparison methods include a parametric or non-parametric binary or M-ary hypothesis test in some of the implementations.

Yet another aspect of the disclosure includes a bioinformatic method for predicting a characteristic of a test cell. The method includes providing an electronic library including non-invasively obtained image data derived from reference cells. The reference cells represent at least two differentiation states and at least two different lineages, and the electronic library further includes molecular data gathered from the reference cells. The method also includes receiving a non-invasively obtained image of the test cell and representing the non-invasively obtained image of the test cell as a multiplicity of pixels. The method also includes deriving image data from the multiplicity of pixels and comparing, by a processor, the image data to non-invasive image data of the electronic library, wherein the processor applies one or more statistical comparison methods to compare the image data. Additionally, the method includes identifying a reference cell that most closely matches the test cell based on the comparison, and predicting that the test cell has a characteristic similar to a characteristic of the identified reference cell, wherein the reference cell characteristic is derived from the molecular data stored in the electronic library in relation to the identified reference cell.

In some implementations of the bioinformatic method, the non-invasive image data is a light micrograph of a living cell and the molecular data is non-image data. In some implementations, the molecular data is characteristic of a cell identity, disease state, or lineage type and includes immunofluorescence data, gene expression data, mRNA and miRNA level data, protein level data, small molecule level data, or enzymatic activity data. In certain implementations, the method also includes verifying whether the test cell has the predicted characteristic.

In another aspect of the disclosure, a method for comparing a test cell to an electronic library of reference cells includes providing an electronic library including non-invasively obtained image data derived from reference cells, wherein the reference cells represent at least two differentiation states and at least two different lineages. The electronic library also includes molecular data gathered from the reference cells. The method further includes receiving molecular data gathered from the test cell and receiving a non-invasively obtained image of the test cell. Additionally, the method includes representing the non-invasively obtained image of the test cell as a multiplicity of pixel and deriving image data from the multiplicity of pixels. Then the image data derived from the multiplicity of pixels is compared to non-invasive image data of the electronic library, wherein the processor applies one or more statistical comparison methods to compare the image data. The method includes comparing the molecular data gathered from the test cell with molecular data gathered from the reference cells, and identifying a reference cell that most closely matches the test cell based on the comparisons of image data derived from the multiplicity of pixels to the non-invasive image data of the electronic library and the molecular data gathered from the test cell to the molecular data gathered from the reference cells.

In some implementations, the non-invasive image data is a light micrograph of a living cell, and the molecular data is non-image data including immunofluorescence data, gene expression data, protein level data, small molecule level data, or enzymatic activity data.

According to another aspect of the disclosure, a method of identifying borders of a cluster of neighboring cells includes obtaining an image of a cluster of neighboring cells; representing the image as a multiplicity of pixels; and segmenting the image using a unified expectation-maximization and level set analysis, thereby identifying the borders of the cluster of neighboring cells.

In some implementations, the cluster of neighboring cells is a stem cell colony, a colony of differentiated cells, or brain tissue. In other implementations, analyzing cell texture includes expectation maximization and level set analysis. In certain implementations, analyzing cell texture comprises performing wavelet decomposition analysis or any multiresolution decomposition algorithm, wherein the wavelet decomposition analysis or multiresolution decomposition algorithm is an n-level decomposition that yields three detail subbands per level.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D are microscopic images comparing pluripotent hESC and differentiated trophectoderm colony images, according to one illustrative implementation.

FIGS. 1E-1F are plots comparing the nuclear area and cell area of pluripotent hESC and differentiated trophectoderm colonies of the cells of FIGS. 1A-1D, according to one illustrative implementation.

FIGS. 1G-1I are plots of empirical probability density functions for three detail subbands, according to one illustrative implementation.

FIGS. 1J-1K are microscopic images of live cells over which spatial frequencies were calculated, according to one illustrative implementation.

FIGS. 1L-1M are plots of spatial frequencies calculated over the white bars of FIGS. 1J and 1K, according to one illustrative implementation.

FIG. 2A is a plot illustrating the statistical dissimilarity measure between segmented windows similar to those of FIGS. 1A-1D, according to one illustrative implementation.

FIG. 2B illustrates iterative classification passes of an image containing a cell cluster similar to those of FIG. 1, according to one illustrative implementation.

FIG. 2C is a detailed view of a micrograph containing a cell colony that has been segmented and classified using the technique of FIG. 2A, according to one illustrative implementation.

FIG. 2D(i)-(iii) are plots illustrating the accuracy of modifying different parameters of the classification technique used in 2A, according to one illustrative implementation.

FIG. 3 is an exemplary output of the classification of stem cell colonies from ten classes, according to one illustrative implementation.

FIGS. 4A-4E are time lapse micrographs of pluripotent stem cell colonies, according to one illustrative implementation.

FIG. 4F is a plot comparing the texture of FIG. 4E to the colonies of FIGS. 4A-4D, according to one illustrative implementation.

FIG. 4G is a plot of KL divergences between each given colony on a given day, and the same or another colony on the same or different days plotted where colonies are clustered by day, according to one illustrative implementation.

FIG. 4H is a plot of the similarity among colonies and between days, according to one illustrative implementation.

FIG. 4I is a plot of the average nuclear area of the cell colonies of FIGS. 4A-4E, according to one illustrative implementation.

FIG. 4J is a plot of the functional changes of the colonies of FIGS. 4A-4E, according to one illustrative implementation.

FIG. 4K is a plot of the lineage markers of the colonies of FIGS. 4A-4E, according to one illustrative implementation.

FIG. 5 illustrates the output at various steps of the Unified EM/Level Set and FMM-EM segmentation processes when classifying a stem cell colony, according to one illustrative implementation.

FIG. 6 illustrates the output at various steps of the Unified EM/Level Set and FMM-EM segmentation processes when classifying brain matter in MR images, according to one illustrative implementation.

FIG. 7 depicts texture filters used for texton generation, according to one illustrative implementation.

FIG. 8 illustrates the output at various steps of the training and segmentation process for stem cell segmentation, according to one illustrative implementation.

FIG. 9 illustrates the output at various steps of the training and segmentation process for stem cell segmentation using the Texture-Based Multi-stage BLS segmentation algorithm, according to one illustrative implementation.

FIG. 10 is a diagram illustrating the components of a system to detect, compare, and classify cells and cell clusters in cell image data, according to one illustrative implementation.

FIG. 11 is a flow chart illustrating a method for identifying the borders of cell colonies using a system similar to the system of FIG. 10, according to one illustrative implementation.

FIG. 12 is a flow chart illustrating a method for comparing cell cultures from different users using a system similar to the system of FIG. 10, according to one illustrative implementation.

FIG. 13 is a flow chart illustrating a method for segmenting and classifying cells and cell clusters using a system similar to the system of FIG. 10, according to one illustrative implementation.

### DETAILED DESCRIPTION

Ex vivo cell culture, especially the culture of stem cells, is a valuable and widely used technique. The visual appearance of cultured cells contains significant information about the cell's identity, including its differentiation status and lineage. However, mere visual inspection of cells is a subjective process subject to inconsistencies between microscopists. This disclosure provides methods of quantifying cells' appearance, allowing automated classification of cells as well as automated delineation of the borders of a cell colony. Also provided are systems and methods for comparing cells cultured by different scientists using different cell culture methods.

The therapeutic promise of pluripotent and adult stem cells derives from their ability to produce tissue-specific cells in a controlled manner. Many applications, such as cell therapy and drug screening, call for extraction of cells and expansion in vitro with defined culture media. Exacting conditions are required to convert all stem cells to the desired lineage with high selectivity over competing lineages. Researchers, regulators and pharmaceutical providers will require new protocols and safety guidelines to ensure production of cells without undesired complications (Xu et al., Circ Res 2002; 91(6):501-8). Production of stem cells for therapeutics is facilitated by adoption of new cell processing and manufacturing methods for quality and safety. Standards for characterizing cell cultures to ensure safety will promote regulatory compliance.

There are typically two preclinical safety concerns for the therapeutic use of stem cells: first is the selectivity and completeness of lineage formation and the second is genomic stability (Xu et al., Circ Res 2002; 91(6):501-8). Both defects are accumulated during in vitro culture and can produce phenotypic changes in morphology and growth rates. Residual pluripotent stem cells will form teratomas after transplantation (Han et al., Neuron 2011; 70(4):626-644; Trosko et al., Toxicology 2010; 270(1):18-34). More insidious, genomic instability acquired during culture might be responsible for tumorigenicity (Baker D E, et al. Nat Biotechnol 2007; 25(2):207-15; Ben-David U, et al Nat Rev Cancer 2010; 11(4):268-277) and mis-targeted cell destinations (Goldring C E P, et al. Cell Stem Cell 2011; 8(6):618-628). Both pluripotent human embryonic stem cells (hESC) (Mayshar Y, et al. Cell Stem Cell 2010; 7(4):521-531) and multipotent adult stem cells (Ben-David U, et al., Cell Stem Cell 2011; 9(2):97-102) are subject to genomic instability in vitro, and promising, induced pluripotent stem cells (iPSCs) currently exhibit the highest rates of chromosomal instability (Ben-David U, et al., Cell Cycle 2010; 9(23):46034604). Genetic defects are selected in vitro that confer a self-renewal advantage and so phenotype characterization can serve as a surrogate biomarker for definitive, but destructive karyotyping of sample cells (Ben-David U, et al., Cell Stem Cell 2011; 9(2):97-102). Close attention to stem cell culture, including design of media and stability of culture environment, will help to maintain a normal karyotype in cell stocks and differentiated progeny.

In addition to quality control for cell therapy, phenotype characterization of stem cells has applications for research, drug development (Cezar G G. Current Opinion in Chemical Biology 2007; 11(4):405-409; Emre N, et al., Current Opinion in Chemical Biology 2007; 11(3):252-258; Sammak P J, et al. In: Hanney S A, editor. High Content Screening: Science, Techniques, and Applications, Methods in Molecular Biology and Enzymology. Hoboken, N.J.: John Wiley; 2008. p 205-224), directed differentiation (Schadt E E, et al., Nat Rev Drug Discov 2009; 8(4):286-295; Chan E M, et al., Nat BiotechnoI 2009; 27(11):1033-7; Hyun I, et al., Cell Stem Cell 2008; 3(6):607-609) and toxicological effects (Davila J C, et al., Toxicol. Sci. 2004; 79(2):214-223; Borowiak M, et al., 2009; 4(4):348-358). Generally, drug discovery and toxicity have been monitored by molecular or phenotypic screens. Each approach has advantages, but a greater number of successful drugs have been identified this past decade by phenotypic screening (Swinney D C, et al., Nat Rev Drug Discov; 10(7):507-19). Therefore phenotypic characterization will have an increasingly important role in identifying successful drugs.

Other applications of phenotype characterization are also of interest. For example, phenotype classification of high throughput production of stem cell colonies and embryoid bodies can be used to assess lineages present in stem cell aggregates (Ungrin M D, et al. PLoS One 2008; 3(2):e1565), teratomas in vivo (Bhagavatula R, et al. Proc IEEE Intl Symp Biomedical Imaging; 2010) and for drug screening in zebrafish embryos (Vogt A, et al. Dev Dyn 2009; 238(3):656-63.) or C. elegans (Gosai S J, et al. PLoS One 2011; 5(11):e15460.). Generally, drug discovery and toxicity have been evaluated at preclinical stages by monitoring molecular targets or more broadly by functional or phenotypic screens. Each approach has advantages, but a greater number of successful drugs have been identified this past decade by phenotypic screening (Swinney D C, et al. Nat Rev Drug Discov; 10(7):507-19).

Pluripotent and adult stem cells in particular are useful for deriving cells that are the most frequent targets for drug toxicity including liver, heart and brain (Davila J C, et al. Toxicol. Sci. 2004; 79(2):214-223.). Differentiation of pluripotent cells can be directed with high efficiency to hepatocytes (Touboul T, Hannan N R, et al. Hepatology 2010; 51(5):1754-65; Sadhana A, et al. Stem Cells 2008; 26(5):1117-1127.) cardiomyocytes (He J-Q, et al. Circ Res 2003; 93(1):32-39; Passier R, et al. Stem Cells 2005; 23(6):772-80; Xu C, et al. Circ Res 2002; 91(6):501-8.), and neurons (Han S S W, et al. Neuron 2011; 70(4):626-644.). Toxicological screening in human cells derived from stem cells (Davila J C, et al. Toxicol. Sci. 2004; 79(2):214-223; Trosko J E, et al. Toxicology 2010; 270(1): 18-34; Guguen-Guillouzo C, et al. Toxicology 2010; 270(1):3-9; Seiler A, et al. Toxicology 2009; 28(2):141-142; Sinha G. Science 2005; 308(5728):1538) has some advantages over animal testing for predicting drug toxicity in humans (Martin M T, et al. Environ Health Perspect 2009; 117(3):392-9; Suter W. Curr Opin Chern Bioi 2006; 10(4):362-6; Vojnits K, et al. Toxicology 2010; 270(1):10-17.). Non-invasive, computer vision-based stem cell classification would advance screening for small molecules that direct development of specific lineages (Emre N, et al. Current Opinion in Chemical Biology 2007; 11(3):252-258; Borowiak M, et al. 2009; 4(4):348-358; Bushway P J, et al. Methods in Enzymology. Volume 414: Academic Press; 2006. p 300-316; Huang A H, et al Ann Biomed Eng 2008; 36(11):1909-21; Ichida J K, et al. Cell Stem Cell 2009; Zong-Yun T, et al. Journal of Cellular Biochemistry 2010; 109(1):93-102; Huangfu D, et al. Nat Biotechnol 2008; 26(7):795-7; Fazzio T G, et al. Cell 2008; 134(1):162-74) or toxicological studies of compounds that inhibit stem cell development (Erb T M, et al. Stem Cells Dev 2011; 20(1):1601-1614; Sinha G. Science 2005; 308(5728):1538; Vojnits K, et al. Toxicology 2010; 270(1):10-17; Seiler A, et al. Reprod Toxicol 2004; 18(2):23140; Chang K H, et al. Biotechnol Bioeng 2004; 88(3):287-98.).

Protocols for producing cells are typically validated by multiple criteria to satisfy regulatory requirements (Goldring C E P, et al. Cell Stem Cell 2011; 8(6):618-628). A complete systems biology approach including phenotype, proteins RNAs and genes enables a network-based understanding of mechanisms of action and requires multiple assays for assessment (Schadt E E et al. Nat Rev Drug Discov 2009; 8(4):286-295). Functional testing such as formation of appropriate tissue after transplantation in vivo is critical for demonstration of developmental capacity. While biochemical staining of representative pluripotency molecular markers including OCT4 are required, detection is destructive and renders the sample unfit for therapeutic use (Sammak P J et al., Methods in Molecular Biology and Enzymology. Hoboken, N.J.: John Wiley; 2008. P 205-224). Live cell fluorescent biomarkers are informative, non-destructive, and have been used to dynamically recognize nascent iPSC colonies (Chan E M, et al. Nat Biotechnol 2009; 27(11):1033-7) but these markers are invasive, requiring genomic modification or membrane permanent dyes that might damage photosensitive cells. Many necessary assays are destructive and so surrogate assays are required to test consistency among batches and facilitate comparisons between protocols (Hyun I, Lindvall et al. Cell Stem Cell 2008; 3(6):607-609).

For particular cell batches intended for therapeutic use or drug development to be characterized without destruction, validated image-based tools to reproducibly quantify amorphous cell and colony morphology in a non-destructive and non-invasive manner are needed. These methods will be of help in quantitatively evaluating large numbers of hESC colonies intended for cell therapy, for producing differentiated progeny at high purity, or for reprogramming iPSCs from somatic cells. A fundamental question concerning visual inspection is under what conditions is cell morphology selective enough to characterize stem cells.

Disclosed herein are method related to image-based assays for phenotypic characterization of stem cells in preclinical stages, where stem cells are expanded and manipulated in vitro before transplantation. Typical visual inspection of phenotype is simple and non-invasive, but is subjective and non-quantitative. Quantitative visual characterization can directly indicate the structural organization of cultured stem cells and cell changes in a characteristic way during self-renewal and differentiation (Erb T M et al., Stem Cells Dev 2011; 20(1):1601-161418). As disclosed herein, in some implementations, colony structure, along with molecular expression profiles, can be viewed as part of a full systems biology approach to phenotype characterization. Therefore, as described herein, phenotype characterization by image-based analysis is useful to provide real-time assessment of cultured stem cells, their culture conditions, and agents that affect cell health and development.

### Exemplary Methods of Segmenting a Cluster of Neighboring Cells

Segmenting methods are used to demarcate different segments within an image. In particular, these methods can be used to segment an image into a cluster of cells and the area outside the cluster of cells, thereby identifying the border of the cluster. There are several contexts in which segmenting methods are useful. First, they can be used to count colonies, for example to quantify the effectiveness of a cell culture condition, the outcome of an experiment, or the result of a transfection or electroporation. In addition, these methods can be used to identify cell colonies prior to computationally classifying the cells. When classifying cells, it is desirable to work from a relatively homogenous image patch, e.g., one that does not contain large areas devoid of cells.

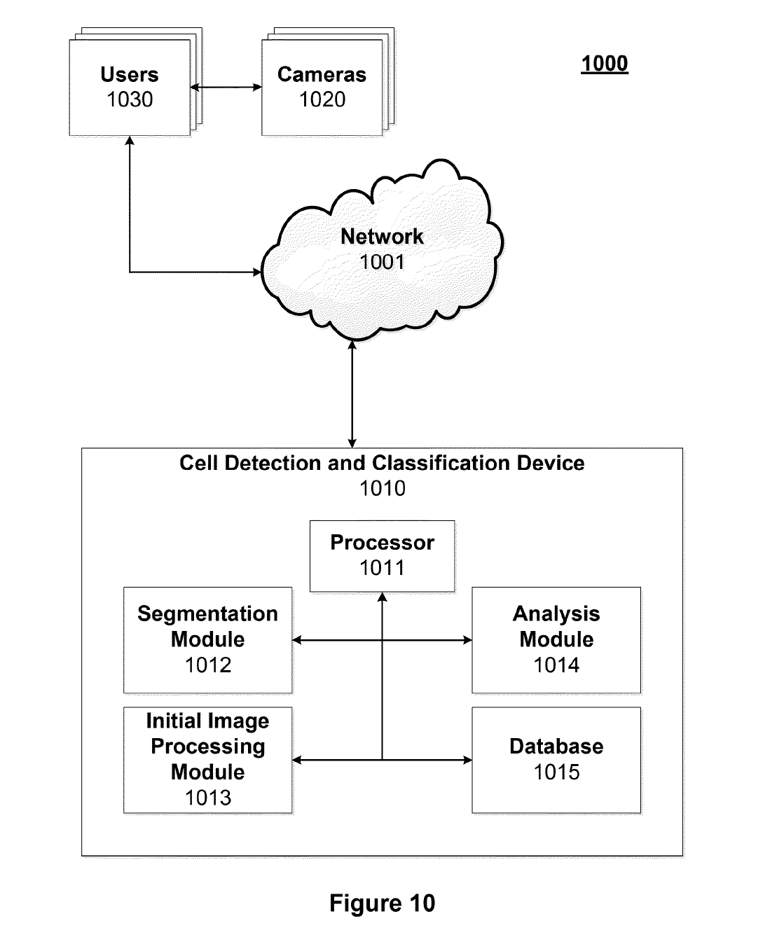

FIG. 10 illustrates a system 1000 for identifying and classifying cell colonies. The system 1000 includes at least one cell detection and classification (CDC) device 1010. The CDC device 1010 receives an image that may contain a cell colony. The processor 1011 of the CDC device 1010 instructs the initial image processing (IIP) module 1013 to perform initial conditioning of the image submitted to the CDC device 101. The segmentation module 1012 segments the image into various segmentation windows for later analysis. Responsive to the generation of the segmentation windows, the analysis module analyzes each window for cells, colony borders, and also classifies each segmentation window. The CDC device 1010 also includes as database 1015 used to store cell and colony classification data. The system 1000 further includes a network 1001 over which users 1030 may access the database 1015 or submit images for cell detection and classification. The images submitted to the CDC device 1010 are captured by the users with cameras 1020.

As illustrated, system 1000 includes a network 1001 that connects users 1030 to the CDC device 1010. In some implementations, the network 1001 includes a local area network (LAN), a wide area network (WAN), wireless area networks, intranets, and other communication networks such as mobile telephone networks, the Internet, any virtual private network or a combination thereof. In other implementations, the network connects a plurality of CDC devices 1010 and users 1030.

The CDC device 1010 receives cellular images. The CDC device 1010 of system 1000 receives cellular images from a camera 1020 via a user 1030. In some implementations, the camera 1020 is a digital camera mounted to a microscope. In other implementations, the camera 1020 is a video camera. In some implementations, camera 1020 captures the image by phase contrast microscopy, fluorescence microscopy, or electron microscopy phase contrast, fluorescence microscopy, luminescence microscopy, magnetic resonance imaging, ultrasound imaging, or widefield microscopy, confocal microscopy, tomographic reconstruction, or statistical reassignment. In some implementations, the camera 1020 is controlled by the CDC device 1010 and/or a user of the CDC device. For example, the CDC device 1010 may be programmed to automatically record a digital image of a cellular colony every 12 hours and detect and classify any cells in the captured image. In other implementations, the CDC device 1010 receives cellular images previously captured. For example, the CDC device 1010 may receive images from a microscope with an attached camera from a collaborator of a user 1030. The received image may originate from a user 1030 that is located in a different physical location than the CDC device 1010 via the network 1001.

The processor 1011 executes various modules having instructions stored on a computer readable medium. In some implementations, the CDC device 1010 includes a plurality of processors 1011. The processors can be single- or multi-core processors.

The IIP module 1013 of the CDC device 1010, performs initial conditioning on an input image. In some implementations, the initial conditioning may include, but is not limited to, cropping the image, reducing or increasing the image pixel density, adjusting the brightness of the image, adjusting the contrast of the image, or any combination thereof. In some implementations, images are reduced to grayscale from the green channel to reduce chromatic aberration and avoid color registration errors.

As illustrated, the CDC device 1010 includes a segmentation module 1012. The segmentation module 1012 receives the image from the IIP module 1013 and then divides the input image into segmentation windows. In some implementations, the segmentation process occurs over several iterations. For example, a large segmentation window of a large size is drawn. If the analysis module 1014 determines the initial segmentation window is non-homogeneous, the segmentation module 1012 sub-divides the initial segmentation window until the analysis module 1014 determines each window is homogeneous.

The analysis module 1014 of the CDC device 1010, analyzes each segmentation window. Described in greater detail below, but briefly, the analysis module 1014 may classify the segmentation windows as belonging to a specific cell type or cell line. For example, the analysis module 1014 may use a Unified Expectation-Maximization and Level Set Approach (UEM/LS) and/or a Texture Based Bayesian Level Set (BLS) Approach to compare the segmentation windows of a image to one another. In other implementations, the analysis module 1014 uses a support vector machine or neural network to classify the segmentation windows. In some implementations, the segmentation windows are classified as growth medium or cellular. In some implementations, the transition between growth medium and cellular section is determined to be a cell colony border. As discussed below in Examples 1, 2, and 3, in some implementations, the cellular segmentation windows are classified based on the type of cell they contain.

The CDC device 1010 also includes a database 1015. In some implementations, the database 1015 includes stored data used to classify and compare cells and/or cell colonies. In some of these implementations, the analysis module 1014 compares stored segmentation windows to the segmentation windows of the captured images to classify the cells and/or colonies of the captured image. Descried in greater detail below, but briefly, in some implementations, the database 1015 contains a library of cellular textures, cell image data, cell culture data, cell condition data, molecular data, or any combination thereof. In yet other implementations, the database 1015 is stored external to the CDC device 1010. For example, the database 1015 is stored on a server that is accessible to the CDC device 1010 and/or users 1030 via the network 1001. In some implementations, the database 1015 is known as a library.

In some implementations, the system 1000 includes a single user 1030, while in other implementations, the system 1000 contains a plurality of users 1030. In certain implementations, the user is a research lab, hospital, research institution, single individual, or any combination thereof.

As descried above, in some implementations, the database 1015 includes image data and non-image data. Image data may include micrographs of cells, for instance obtained by light microscopy or electron microscopy. The light microscopy images may be obtained by, for instance, phase contrast, brightfield, or darkfield microscopy. The images may also be collected using fluorescence microscopy, for instance using immunofluorescence, fluorescent chemical dyes, or fluorescent proteins. In some implementations non-image data is data derived from cells that does not include an image of the cells, regardless of whether the data can be presented in graphical form. Non-image data includes nucleic acid (e.g., DNA or RNA) levels determined by, e.g., microarray or quantitative PCR, protein levels determined by, e.g., Western blot or ELISA, enzymatic activity measurements, or measurements of small molecules such as metabolites or even ions.

In some implementations, molecular data includes non-destructive or destructive biomarkers. For example, in some implementations, the data stored in the database 1015 is gathered non-invasively from live cells without significantly altering the cells. In some implementations, the database includes molecular data that is gathered invasively. For instance, the molecular data may be gathered through a method that requires fixation or disruption of the cells. Molecular data includes immunostaining or immunofluorescence data, in situ hybridization data, data about protein levels (e.g., determined by Western blot or ELISA), and data about gene expression (e.g., gathered by microarray or quantitative PCR).

In some implementations, database 1015 includes image data sets of cells collected from the same cell colony at different time points (for instance before and after an episode of treatment). In certain implementations, the first cell is a cell treated with a test agent (such as a drug or toxin) and the second cell is a control cell not treated with the test agent. In some implementations, the first cell is intended for use in cell therapy, and the second cell is a reference cell that represents the desired phenotype for the therapeutic cell.

In some implementations the database 1015 includes multiple images of the same cells or same culture of cells. For instance, the database 1015 may contain images gathered across a time course experiment, such as a differentiation time course. The images may be gathered across different time scales. For instance, the database 1015 may include time course data gathered at least every 1, 2, 6, 12, 24, or 48 hours. The database 1015 may also include time course data gathered over the course of at least 1, 2, 5, 10, 15, or 20 days. As an additional example, the database 1015 may contain images having different spatial resolution. For instance, the database 1015 may contain images gathered using different fold magnification using different microscope lens objectives. In some implementations, the data contains images gathered using at least 2, 3, 4, or 5 different magnifications.

In yet other implementations, the database 1015 includes a texture library. In some implementations, the database 1015 includes at least 5, 10, 15, or 20 textures. In some implementations, database 1015 includes data from fibroblasts, mouse embryonic fibroblasts, human stem cell, or any combination thereof. In yet other implementations, the database 1015 includes cell data relating to cells intended for tissue engineering, cells intended for cell therapy, cells relating to a specific disease or condition, or any combination thereof.

In some implementations, the system compares the cell image data and/or molecular data to the library to determine, for example, differentiation state, differentiation rate, and/or eventual cell type, health, viability, etc. In some implementations, a particular reference library (e.g., stem cells induced to differentiate by a given method) includes about 10, 50, 100, 150, 200, 250, 300, 350, 400, 450, 500, 550, 600, 650, 700, 750, 800, 850, 900, 950, 1000, 2000, 3000, 4000, 5000 or more images. In some implementations, the reference library additionally includes non-image data.

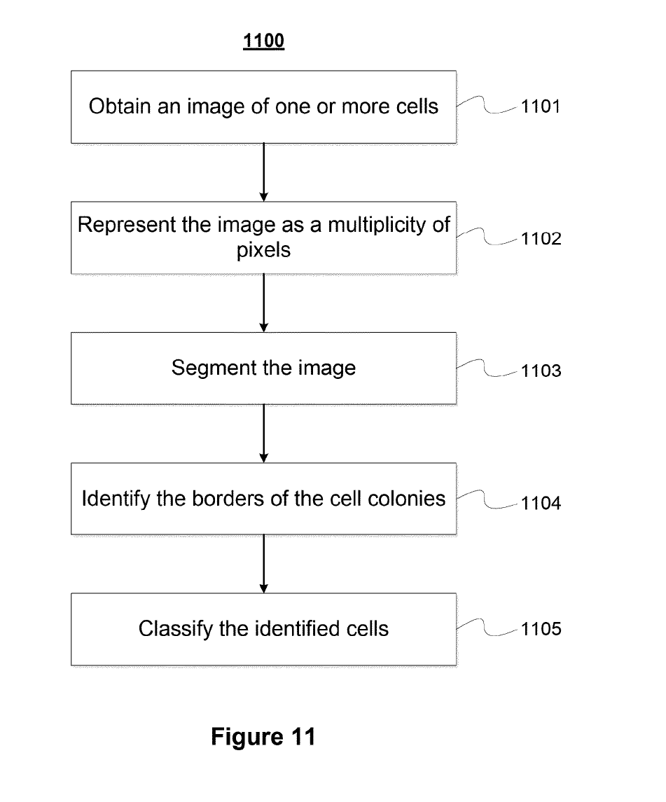

FIG. 11 illustrates a method 1100 of classifying cells in a cluster of neighboring cells using system 1000. The method 1100 begins by obtaining an image of a cluster of neighboring cells (step 1101). Next, the image is represented as a multiplicity of pixels (step 1102). Responsive to being presented as a multiplicity of pixels, the image is segmented (step 1103) and the borders of the cluster of neighboring cells are identified (step 1104). The method 1100 concludes with the classification of the identified cells (step 1105).

As set forth above, the method 1100 of identifying borders of a cluster of neighboring cells includes obtaining an image of a cluster of neighboring cells (step 1101). In some implementations, the image is obtained with a camera 1020. In some implementations, the camera is any video or imaging camera capable of resolving cellular colonies. In some implementations, a plurality of images are captured and the images are analyzed individual and/or the differences between the images are analyzed.

Responsive to the image being captured, the method 1100 continues by representing the image as a multiplicity of pixels (step 1102). In some implementations, the pixel density of the captured image is altered. For example, the pixel density can be increased or decreased. In some implementations, an image filter is applied to remove image artifacts, filter the image, prepare the image for later analysis, or any combination thereof.

The method 1100 of identifying borders of a cluster of neighboring cells continues by segmenting the image into a plurality of segmentation windows (step 1103). The segmentation and identification steps are described in greater detail below and in more specifically in Examples 2 and 3, but briefly, in certain implementations, segmentation is performed using a unified method combining Expectation-Maximization and Level Set (EM-LS) analysis. In other implementations, segmentation is performed using multi-stage Bayesian Level Sets. For example, in some implementations, the unified EM-LS analysis includes subdividing the captured image and analyzing the cell texture in each of the subdivided segments. In yet other implementations, the cell texture analysis includes any statistical or non-statistical wavelet decomposition and/or multi-resolution decomposition analysis. In yet other implementations, the captured image is segmented and classified using other texton analysis methods.

In some implementations, the texton analysis methods include calculating a texton for each of a plurality of pixels, and comparing these textons to a library of textons. A texton is a vector comprising a plurality of filter responses derived by filtering a given pixel. The library of textons may include textons derived from cells of known type (e.g., species, differentiation status, and lineage).

In some implementations, the segmentation method does not require a user to manually identify the cluster of neighboring cells. Certain segmentation methods require the user to draw a box or other approximate border around a cell colony; in some aspects, the segmentation methods herein do not require this step.

After segmenting the image, the method 1100 continues by identifying the borders of the cell colonies (step 1104). In some implementations, the borders are identified as the transition from one segment to another segment. In some implementations, defining a border includes determining if the segment is homogeneous. For example, if the texture of the segment is determined to not be homogeneous, the segment may be resegmented until each segment is homogeneous.

The method 1100 concludes with the classification of the identified cells (step 1105). In some implementations, the classification of the identified cell includes comparing the texton of each segment to a library of reference textons. In some implementations, the segmentation step 1104 is performed prior to the cell classification step 1105. In other implementations, the classification step 1105 and the segmentation step 1104 are performed simultaneously.

### Exemplary Texture Analysis for Phenotype Characterization

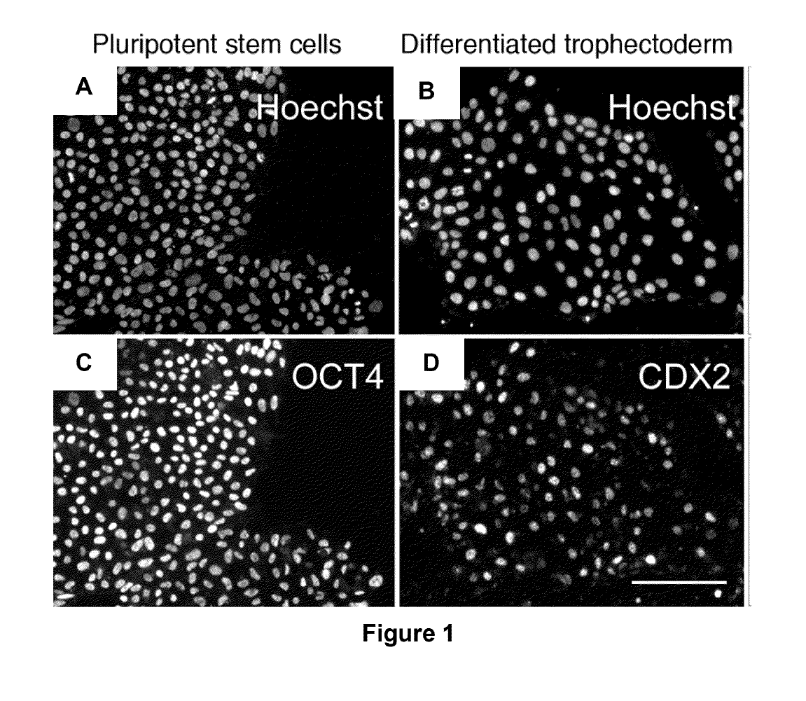

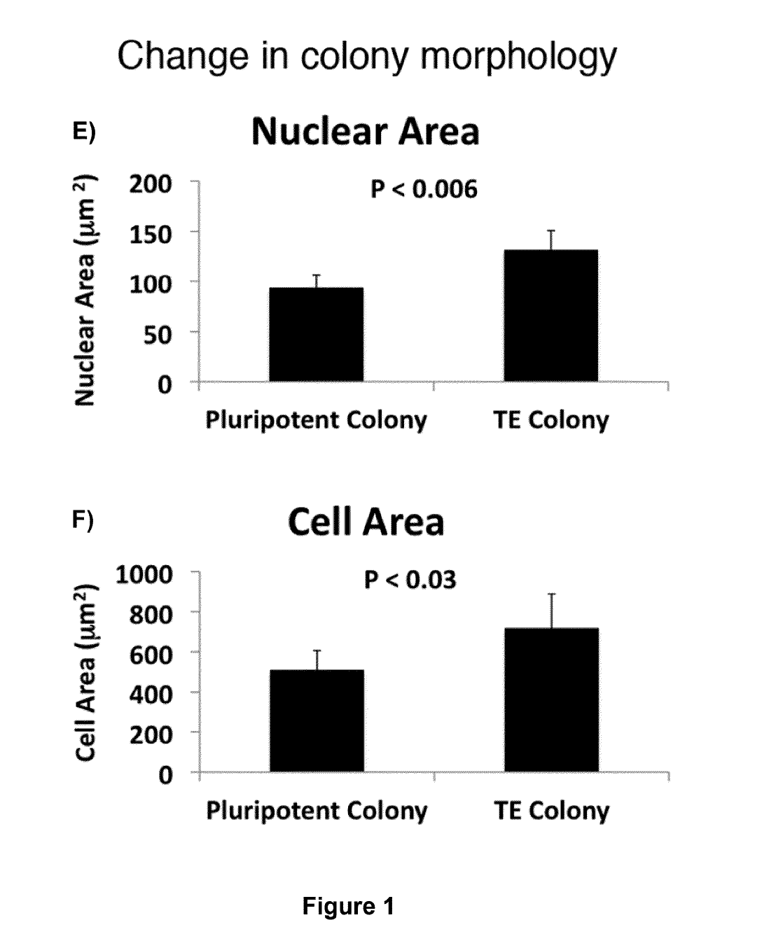

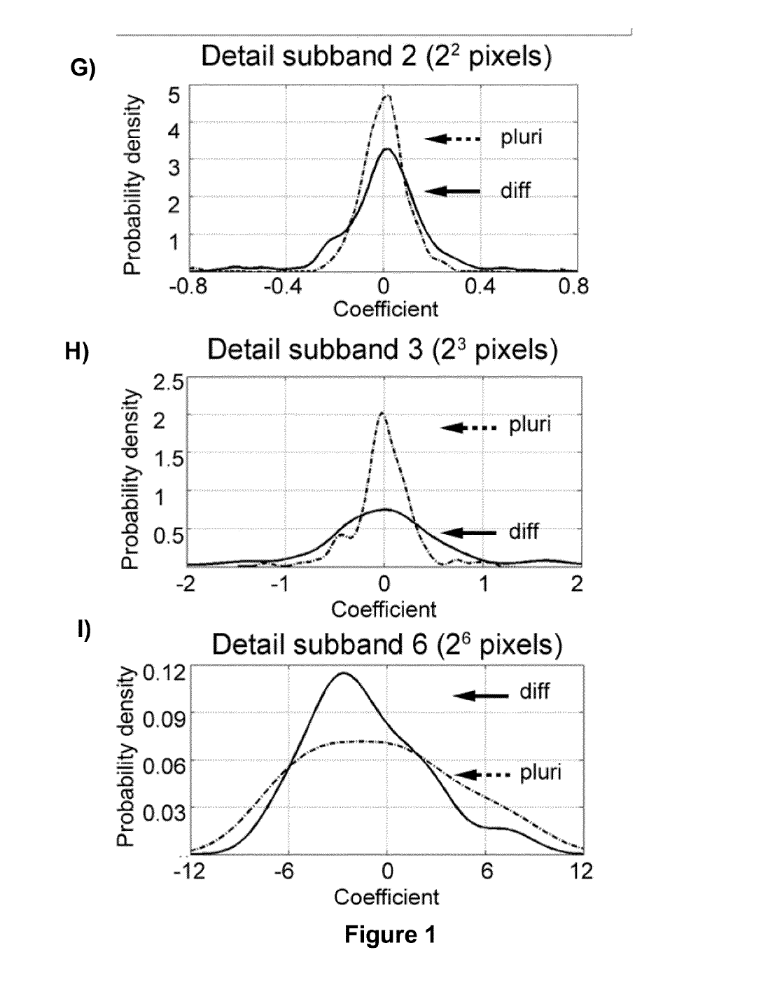

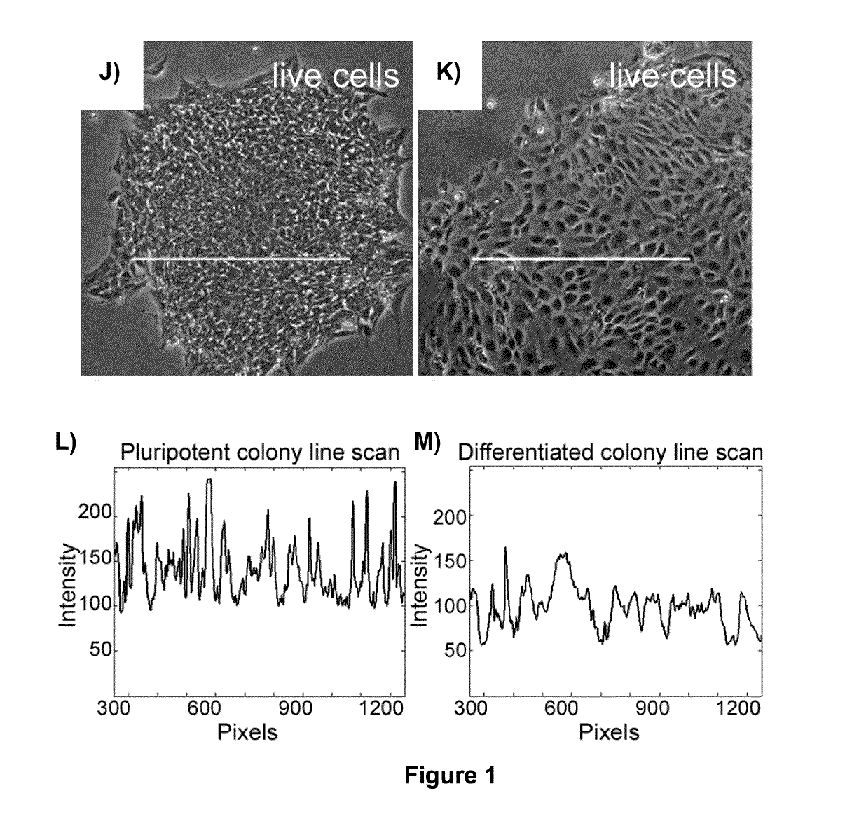

Texture analysis is a quantifiable method for measuring amorphous, non-geometric properties of cell morphology, particularly nuclear and cytoplasm size and edges (see e.g., FIG. 1). Because structure often follows function, different cell lineages often, but not always, have characteristic cell size, shape, and appearance, making cell morphology one important indicator of phenotype. Morphology combined with multiple molecular markers is typically required to fully characterize cellular phenotype.

Disclosed herein are methods for distinguishing cell lineage based on texture analysis. Computer vision is an aid at the disposal of the experienced microscopist and extends human capacity by being able to reproducibly categorize large amount of data. In the case of stem cell differentiation, the number of possible lineages is large, but not unmanageable, and as disclosed herein, texture, as validated with canonical molecular biomarkers serves as a sufficient non-invasive surrogate or alternate biomarker for molecular lineage markers during directed differentiation.

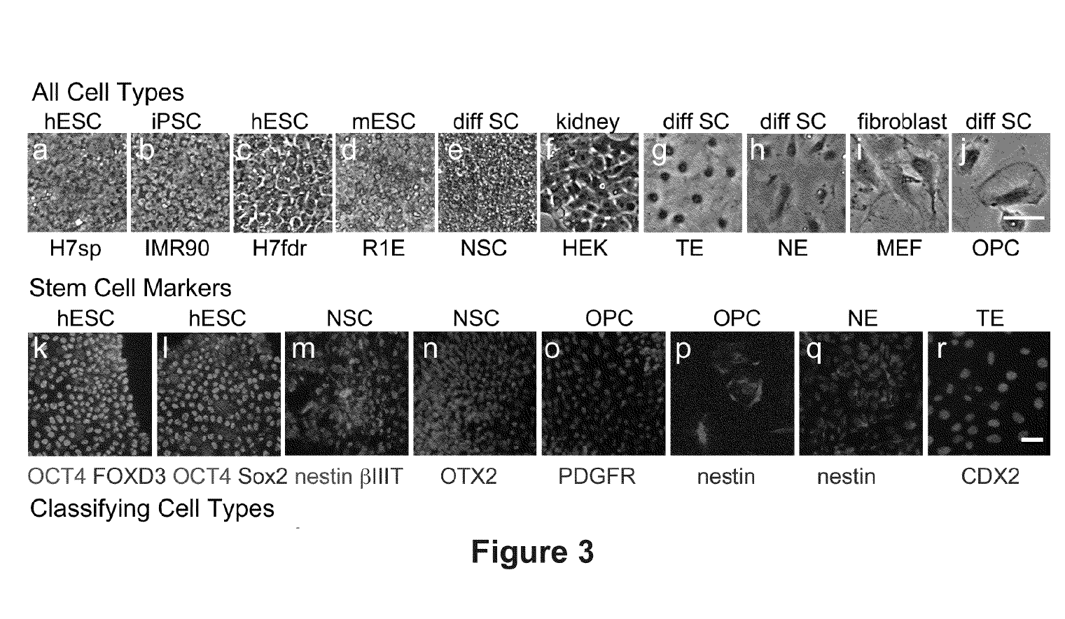

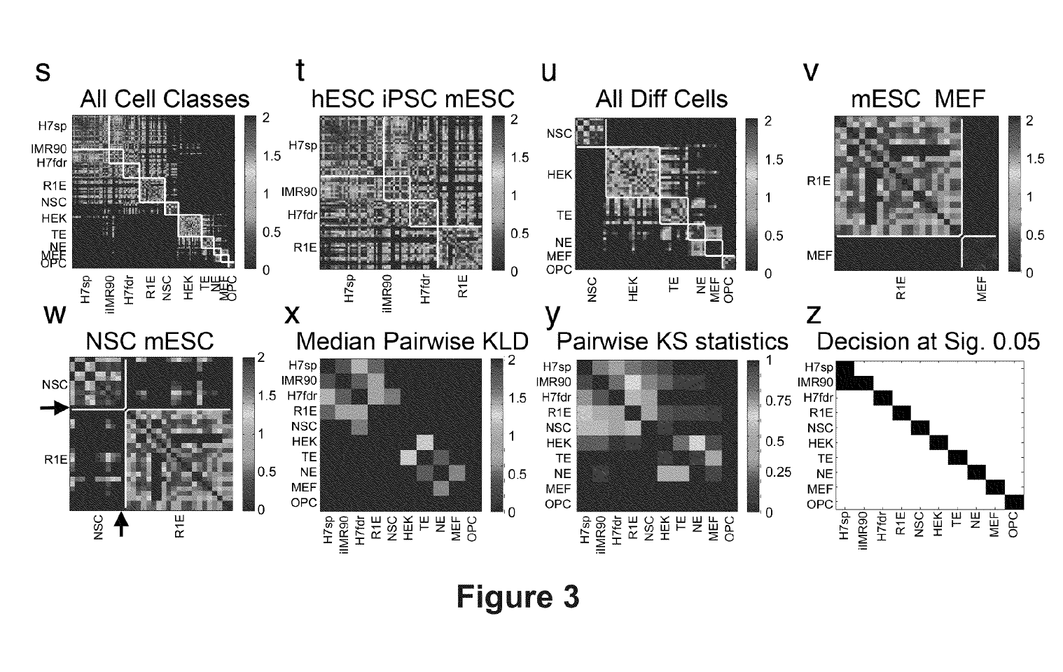

Pluripotent cells exist in different functional states. By way of example but not by way of limitation, mouse and human pluripotent stem cells have characteristics of the pre-implantation embryo's inner cell mass or the post implantation epiblast, respectively, and the inner cell mass expresses higher levels of pluripotency markers (Toyooka Y, et al. Development 2008; 135(5):909-18; Tesar P J, et al. Nature 2007; 448(7150):196-9; Brons I G M, et al. Nature 2007; 448(7150):191-195; Nichols J, et al. Cell Stem Cell 2009; 4(6):487-492). These states are metastable and require treatment for interconversion (Xu Y, et al. PNAS 2010; 107(18):8129-8134; Zhou H, et al. J BioI Chern 2011; 285(39):29676-80). As another example, reduced OCT4 levels can be compensated by increased cellular junctions containing E-cadherin (Redmer T, et al. EMBO Rep 2011; advance online publication), suggesting that colony morphology has a direct role in pluripotency. These distinct pluripotent states have different developmental potentials for extraembryonic lineages (Erb T M, et al. Stem Cells Dev 2011; 20(1):1601-1614.) and as shown herein, can be distinguished by texture-based phenotype characterization (see e.g., FIG. 3 and Example 1).

In some implementations, in addition to wavelet decomposition, other image-based methods can be applied for non-invasive analysis. For example, in some implementations, automated pattern recognition software tools are used, which depend on training sets rather than defined algorithms for classifying biological images (Swedlow J R, et al Annual Review of Biophysics 2009; 38(1):327-346). For example, in some implementations, Wnd-Charm, available within the Open Microscopy Environment, is a weighted, software tool that can be applied to unsegmented images for classification with 1025 output features based on high contrast, pixel statistics and texture transforms, (Shamir L, et al. Source Code Biol Med 2008; 3:13; Odov N, et al. Pattern Recognit Lett 2008; 29(11):1684-1693). In some implementations, useful features from the 1025 image-feature set are texture features, which compare favorably to task-specific algorithms (Shamir L, et al. Source Code Biol Med 2008; 3:13). In addition, transforms may be used in some implementations, including Chebyshev applied in combination with secondary transforms (Odov N, et al. Pattern Recognit Lett 2008; 29(11):1684-1693), or curvelet transforms, a wavelet variant that is less dependent image directionality (Zhang B, et al. BMC Bioinformatics 2011; 12(1):128). In one implementation, e.g., as discussed in Example 1, wavelets were evaluated for the particular application of discriminating phase contrast images of human stem cells using validated libraries and demonstrated strong performance. This approach captures two characteristics of texture: change in intensity that occurs i) at multiple scales and that ii) has random characteristics.

### Exemplary Characterization of Structure, Phenotype and Function

In some implementations, inspection by computer vision is used to complement molecular characterization. Computer vision inspection is a non-destructive measure of whole-cell structure and function and in some implementations, it can be applied to the whole population, identifying outlier behavior. In some implementations, periodic inspection can also be used to better characterize dynamic phenotypes such as self-renewal or differentiation. The ability to evaluate cultures for dynamic or reversible changes is useful, for example, for quality control of samples destined for cell therapy and for rapid screening of drugs using higher-order phenotype to capture off-target cellular effects.

In some implementations, the image based methodology disclosed herein serves as a statistical and quantitative support aid for biologists and clinicians engaged in growing, maintaining, and analyzing hESCs or iPSCs, with the potential for automated image acquisition and analysis to autonomously assess and monitor the degree of pluripotency in a non-destructive manner. As disclosed herein, quantitative texture-based statistics are useful as a non-invasive, non-destructive biomarker for phenotype characterization.

### Exemplary Quantifying Comparisons Between Cells

In certain aspects, the system 1000 and detection and classification methods disclosed herein provide bioinformatics tools for quantitative image comparison. In some implementations, the system 1000 and methods allow cell biologists to evaluate their protocols against established protocols and quantitatively assess the effect of protocol modification. The statistical bioinformatics methods herein allow for non-invasive stem cell phenotyping and provide a frame of reference, common platform, and statistical language that enable more precise and fruitful comparison of cells and culture conditions.

In some implementations, the methods compare cells cultured using similar protocols performed by different users. In some implementations, the methods compare cells cultured using different protocols performed by different users in order to determine the impact of the difference between the protocols. In some implementations, the methods are used for quality control to assist promote culturing techniques between different users. In some implementations, the cells are stem cells, for instance for use in stem cell therapy.

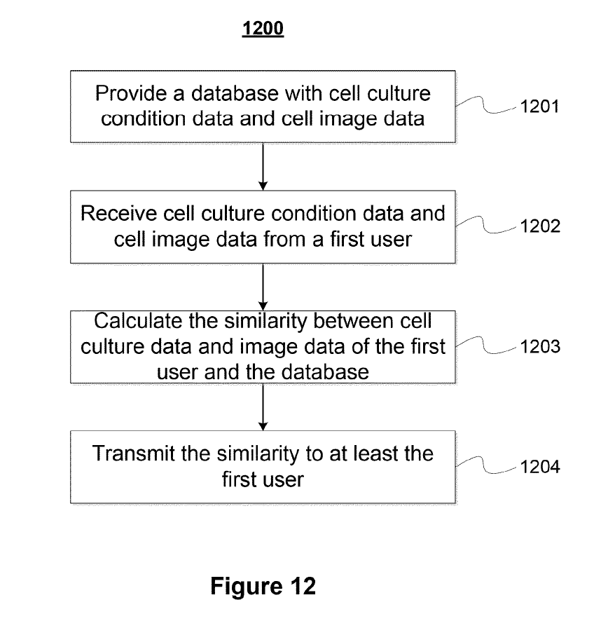

FIG. 12 is a flow charge of a method 1200 for comparing cells cultured by a user to an electronic library of cells. The method 1200 includes providing a database suitable for storing cell culture condition data and cell image data (step 1201). The method 1200 also includes receiving cell culture condition data and cell image data provided by a first user (step 1202). Responsive to receiving the data from the first user, the similarity between the data provided by the first user and the data of the electronic library is calculated using one or more statistical comparison method (step 1203). The method 1200 concludes with the transmission of the previously calculated similarity to at least the first user (step 1204).

As set forth above and described in relation to system 1000, the method 1200 begins by providing a database suitable for storing cell culture condition data and cell image data (step 1201). In some implementation, the database is the database 1015 of the CDC device 1010. In some of these implementations, the database is made accessible via the network 1001. In yet other implementations, the database resides on a central server, and in other implementations, the database resides on a local computer. In certain implementations, the database resides over a plurality of computers. For example, cell culture data may reside on a first server and cell image data may reside on a second server.

The method 1200 of comparing cell cultures continues with receiving cell culture condition data and cell image data from a first user (step 1202). In some implementations, the method includes comparing more than two sources of cell culture image data. For instance, at least 3, 5, 10, 15, 20, 50, or 100 or more different images are compared. In some implementations, a second user also submits cell culture data and cell image data to the database for comparison.

Responsive to receiving data from a first user, the method 1200 continues by calculating the similarities between the data provided by the first user and the data of the library (step 1203). In some implementations, calculating the similarities between the user's data and the data of the library allows a user to predict a molecular characteristic of a cell based on non-invasively gathered data. In some implementations, the calculation of the similarity between cell image data provided by the users allows the users to compare image data and/or non-image data derived from cells. In certain implementations, the cell image data and cell condition data of the first user is compared to the cell image data and cell condition data of the second user. In yet other implementations, the cell image data and cell condition data of the first and/or second user is compared to the cell condition data and cell image data of the database.

In some implementations the step 1203 of calculating the similarities between the user's data and the library's data is achieved by a statistical comparison of the data. The statistical comparison method used to compare the cell condition data and/or image data from different sources includes, but is not limited to, a comparison of normalized or unnormalized probability density functions or estimates thereof, including a subset of the infinite number of moments a distribution can have; a parametric statistical method; a non-parametric statistical method; a M-ary hypothesis test; or any combination thereof. In some implementations, the similarity is quantified using information divergence and/or relative entropy.

In certain implementations, the database includes a collection of established cell culture protocols. Accordingly, in some implementations, the step 1203 of calculating the similarity between the user's data and the library's data includes performing a text comparison between two protocols to identify differences between them.

In some implementations, calculation step 1203 compares non-invasively gathered data and molecular data. For instance, the system may compare phase contrast image data (which is non-invasively gathered image data) and differentiation marker expression data (which is molecular data, and may be image data or non-image data) corresponding to a first cell culture method with phase contrast image data and differentiation marker expression data corresponding to a second cell culture method. In some implementations comparing non-image data and/or molecular data includes, e.g., calculating the fold difference between the level of a type of molecule in a first sample versus the level of the same type of molecule in the second sample. In some implementations, the levels of a molecule of interest are normalized to a control, such as a housekeeping gene or protein.

In other implementations, the calculation step 1203 also includes information about the cell sample (e.g., source organism, tissue type, starting cell type, etc.). When such information is provided, the system may optionally compare the test cell only to reference cells (e.g., by comparing image data, non-image data, and/or textons) associated with the same or similar cultures. For example, if a user specifies that the starting cells are mouse embryonic stem cells, the system may compare test images only to other vertebrate (or mammalian or even mouse) cells, other stem cells or not-fully differentiated cells or some union set of both types of cell categorization.

In yet other implementations, the calculation step 1203 compares the user's data to specific data in the library. In some implementations, the specific data in the library is selected by the user. For example, the specific data may be a specific cell, cell line, or cell type. As a further example, the user may wish to compare a cell colony with the cell colony of a collaborator.

The method 1200 concludes by transmitting the similarity between the user's data and the library's data to at least the first user (step 1204). In some implementation, the comparison of image and cellular data to the data of the database is used to make predictions about molecular data. In some of these implementations, the prediction is also transmitted to the user. For instance, in some implementations the system 1000 determines that an uncharacterized cell resembles a reference cell in the database based on non-invasively gathered image data, and predicts that the uncharacterized cell has molecular data (e.g., gene expression data) similar to that of the reference cell.

In some implementations, the comparison of cellular data is used in feedback modification of cell culture conditions. For example, researchers sometimes wish to improve a cell culture protocol to achieve a high proportion of a desired type of cell. The user may, for example, test several variations of a starting cell culture condition and then quantify the similarity between cells obtained via those conditions versus reference cell. The user can then select the variant condition that produces cells most similar to a desired reference. In other implementations, the user may perform several iterations of varying cell culture conditions, quantifying cell phenotypes, thereby identifying a desired cell culture condition. The user may perform, e.g., 1, 2, 3, 4, 5, or more iterations.

In some implementations, the transmission step 1204 also includes providing the user with a listing of at least one cell or cell line most similar to the reference cell that produced the data initially provided by the user in step 1202. For instance, the 2, 3, 5, 10, or 20 most similar cells of the database may be identified and provided to the user.

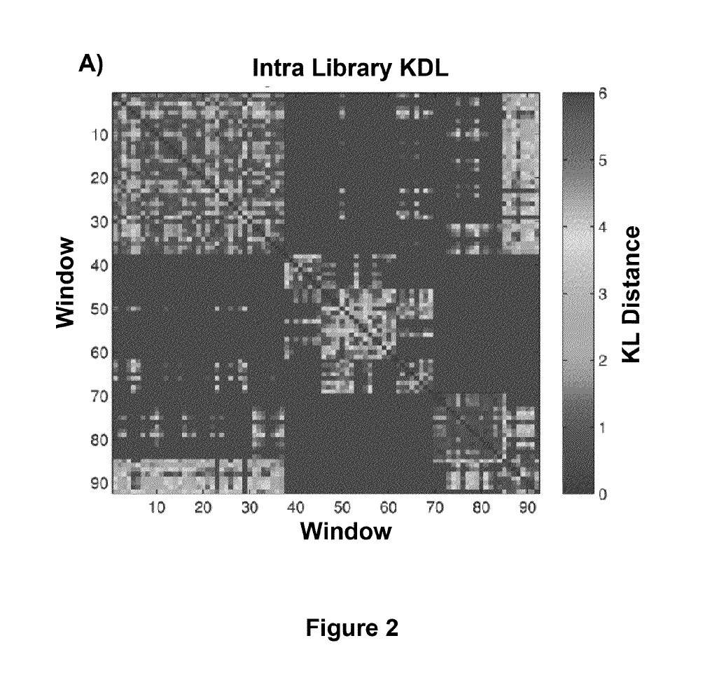

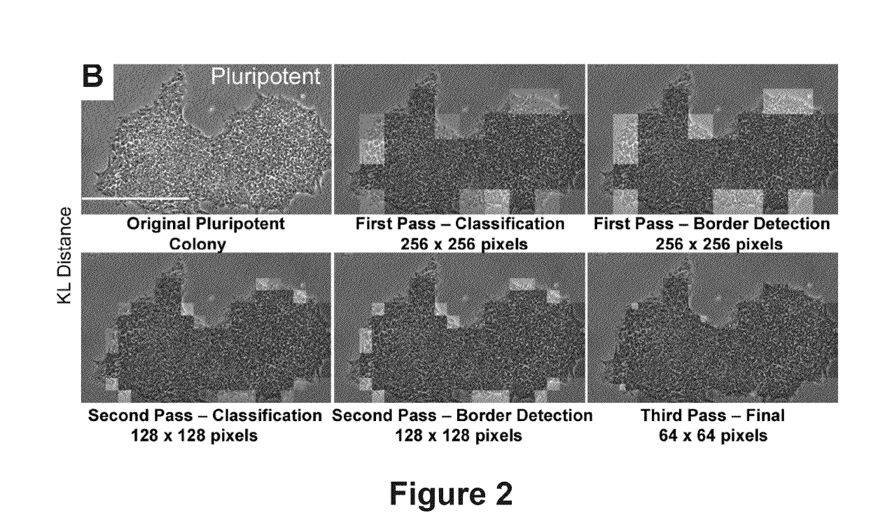

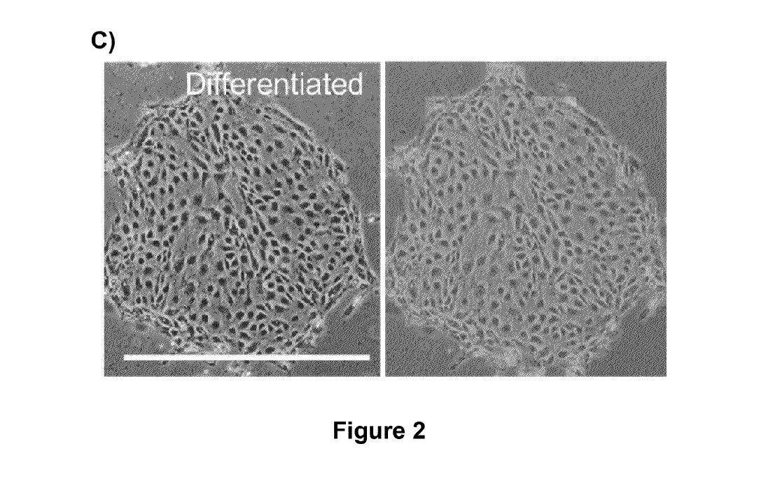

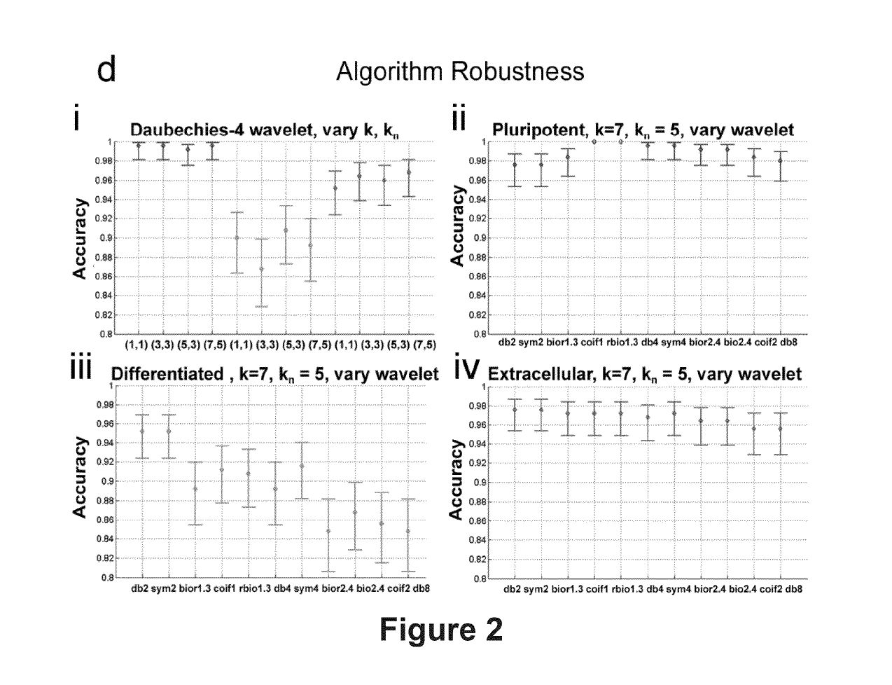

In some implementations, the similarity between the first cell image data set and the database of image data sets is transmitted to one of the users via the network 1010. In some implementations, the results are transmitted and displayed to the user via a custom graphical representation. For example, the custom graphical representation can have elements similar to the plots showing below in relation to FIGS. 1 and 2.

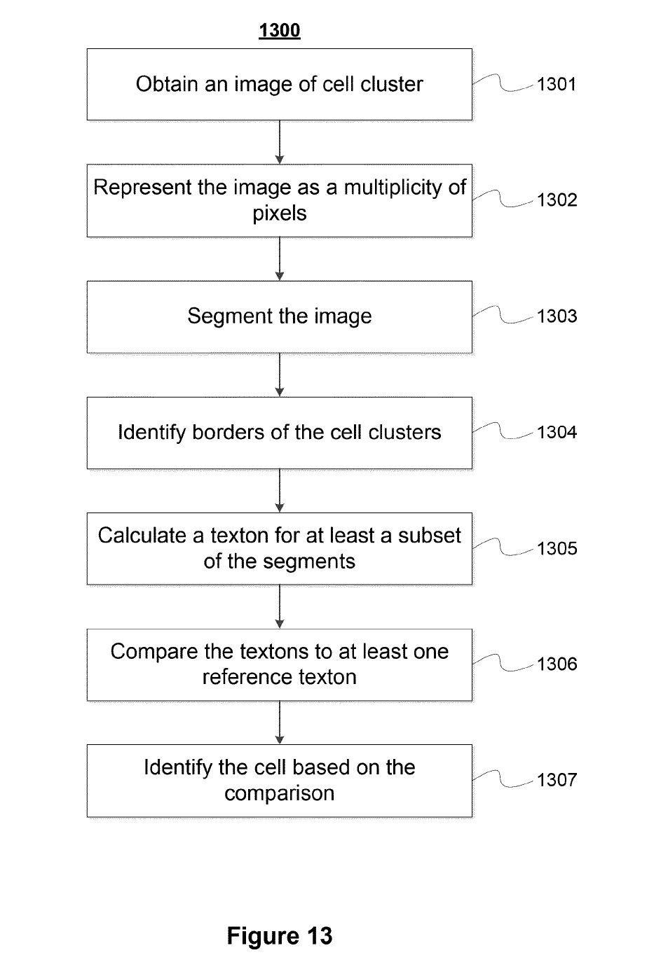

FIG. 13 illustrates a method 1300 for segmenting and then classifying images of cell clusters by system 1000 as can be used in methods 1100 and 1200. The method of segmenting and classifying cell cluster images includes obtaining an image of a cell cluster (step 1301). The image of the cell cluster is represented as a multiplicity of pixels (step 1302) and then segmenting (step 1303). Next, the borders of the cell clusters in the segmented image are identified (step 1304). Responsive to being identified, a texton is calculated for at least a subset of the segments (step 1305). Then the calculated textons are compared to at least one reference texton (step 1306). Based on the comparison of the calculated textons to the at least one reference texton, the cells of the cell cluster are identified (step 1307).

As set forth above and referring to FIG. 10, the method 1300 of segmenting and classifying images of cell clusters begins with obtaining an image of a cell cluster (step 1301). As described above in reference to system 1000, in some implementations, the image is received directly from a camera 1020. In other implementations, the image is received from a first or second user.

Upon receiving the image, the captured image is represented as a multiplicity of pixels (step 1302). In some implementations, the captured image is a digital image and is initially received by the system 1000 as a multiplicity of pixels. In other implementations, the captured image is transformed to have a specific pixel density by a initial image processing module 1013. In certain implementation, the initial image processing module 1013 also performs additional initial transformations such as those descried above. For example, the initial image processing module 1013 may crop the captured image so that it conforms to a specific size.

The method 1300 continues by segmenting the captured image (step 1303). Described in greater detail in Examples 2 and 3, in some implementation, the captured image is segmented with BLS analysis or a unified EM-LS analysis. In some implementations, the system 1000 defines portions of the captured image that contain cells from portions of the image that predominately contain growth medium or/and or dead cells. In some implementations, the system 1000 also identifies segments devoid of cells that are within larger segments containing cells. For example, the CDC device 1010 may identify the captured image has a large cell cluster in the middle of the captured image; however the CDC device 1010 may also determine that a segment in the center of the cell cluster contains dead or no cells.

Responsive to segmenting the image, the method 1300 identifies the borders of the cell clusters (step 1304). In some implementations, the borders are defined as the transition from one segment to another segment. In certain implementations, identified borders are checked by a user before the method continues. In yet other implementations, the method 1300 is completely automated and devoid of input from a user.

After identifying different segments in the captured image, the method 1303 continues by calculating a texton for at least a subset of the segments in the captured image (step 1305). In some implementations, such as Example 3, the textons are calculated during the segmentation step 1303. In other implementations, such as Example 2, the captured image is segmented using the unified expectation-maximization and level set analysis and the textons are not calculated until the segmentation process is complete. In some implementations, calculating the texton includes calculating at least eight filter responses. In other implementations, the calculation of the texton includes using a Gaussian filter, a Laplacian-of-Gaussian filter, or a bar filter.

The method 1300 continues by comparing a calculated texton to at least one reference texton (step 1306). Discussed in greater detail in Example 3, but in general, the calculated texton is compared to the at least one reference texton with a statistical comparison method. For example, the statistical comparison method may be a parametric, non-parametric, or M-ary hypothesis tests. In some implementations, the reference texton is included in the database described above.

The method 1300 concludes by identifying the cells of the cell cluster (step 1307). In some implementations, the identification of the cells in the cell cluster is determined to be the cell type that generated the reference texton having the greatest statistical similarity with the cells of the cell cluster. In some implementations, the reference cells include, but are not limited to mouse cells, human cells, embryonic stem cells, induced pluripotent cells, neural stem cells, kidney cells, trophectoderm cells, neurectoderm cells, fibroblasts, oligodendrocyte precursor cells or any combination thereof.

The following illustrative examples provide further detail regarding the segmentation, analyzation, and classification of cell image data as descried above. These specific examples are included merely to illustrate certain aspects and implementations of the present invention, and they are not intended to limit the invention in any respect. Certain general principles described in the examples, however, may be generally applicable to other aspects or implementations of the disclosure. Any features or implementations described above and below can be combined.

### EXAMPLES

### Example 1

### Non-Invasive Characterization of Stem Cell Phenotype Using Image Texture Analysis

### A. Introduction

Stem cells used in drug screening, tissue engineering, and cell therapy need reliable nondestructive characterization to ensure safety and efficacy. Most researchers use visual microscopic inspection to monitor the phenotype of self-renewing or differentiating stem cells without perturbing the cell culture, but for definitive characterization lineage-selective molecular markers are usually applied to subsamples that are sacrificed for that purpose. In this example, computer vision methods are applied, such as multiresolution statistical image analysis, to reproducibly classify the structural organization of cells and colonies in a non-invasive non-destructive manner. In this example, non-Gaussian statistical analysis of an image's wavelet decomposition provides non-destructive, automatable and real-time classification of the texture of stain-free colonies. An iterative, hierarchical windowing approach separates areas with different textures and a machine learning approach establishes a validated reference library of textures to characterize unknown developmental phenotypes. Each reference developmental phenotype in the texture library was validated with immunofluorescent staining and high content screening of a panel of molecular linage markers. Pluripotent human embryonic stem cells (hESCs) were classified by texture with 99% success while all 9 developmental phenotypes tested were distinguished at 95% confidence levels. Pluripotent mouse and human ESCs, multipotent stem cells including trophectoderm, neurectoderm, neural stem cells, and oligodendrocyte precursor cells, fully differentiated cell types including somatic epithelial cells and fibroblasts were evaluated. Furthermore, time-lapse imaging of hESCs showed distinct phenotypes during colony growth and compaction that can also be distinguished by cytoplasmic actin structure, and by lineage markers. Cell and colony structure, along with molecular expression profiles, form part of a systems biology approach to phenotype characterization. Therefore, as shown in the example, phenotype characterization by texture analysis can assist in real-time assessment of cultured stem cells, their culture conditions, and agents that affect cell health and development.

Multiresolution statistical texture analysis is an effective means of retrieving and classifying textural data (Do M N et al., IEEE Trans Image Processing 2002; 11(2):146-58). Successful biological applications of multiresolution texture analysis include fluorescent images of protein distribution in cells (Chebira A et al. BMC Bioinformatics 2007; 8:210) and nuclear texture characteristic of aggressive cancers in patient biopsies (Qureshi H et al., Med Image Comput Comput Assist Interv 2008; 11(2):196-204; Weyn B et al., Cytometry 1998; 33(1):3240). As illustrated in this example, these analytical approaches are applied to stem cell colonies, where apparent texture corresponds to morphological variation indicative of pluripotency, maturity, and differentiation state (Jeffreys C G. [Thesis (S.M.)]. Cambridge, Mass.: Massachusetts Institute of Technology; 2004; Mangoubi R, et al., Proc IEEE Intl Symp Biomedical Imaging; 2008; Mangoubi R, et al., Proc IEEE Intl Symp Biomedical Imaging; 2007.). As shown in this example, multiresolution image-based statistical texture analysis enables the quantification of real-time classification of hESC colonies in phase-contrast microscopic images.

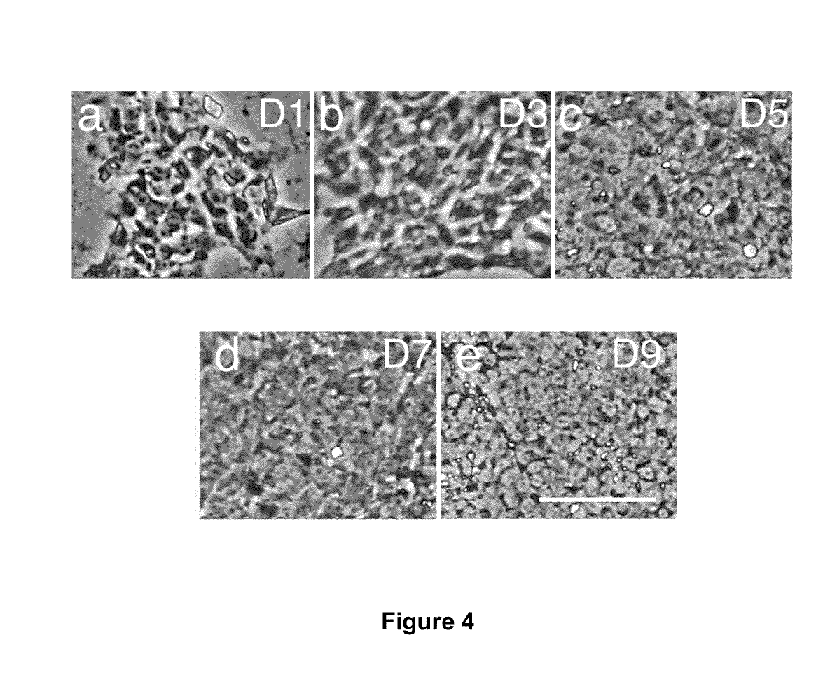

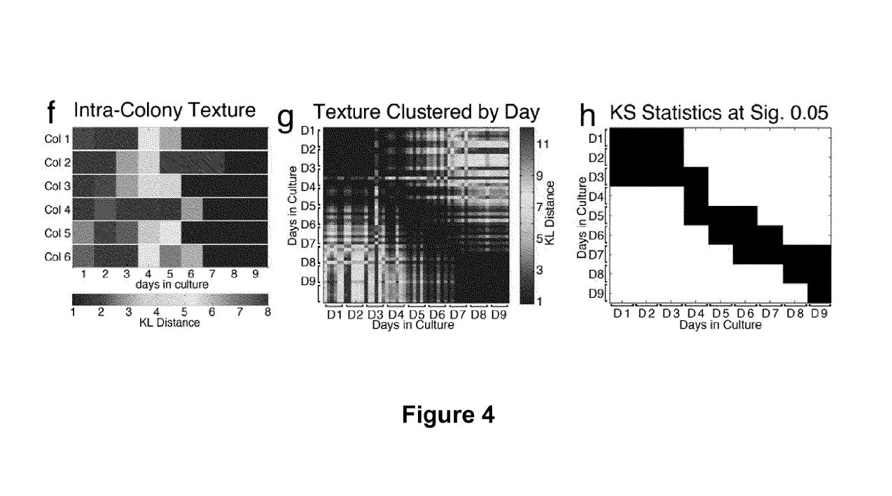

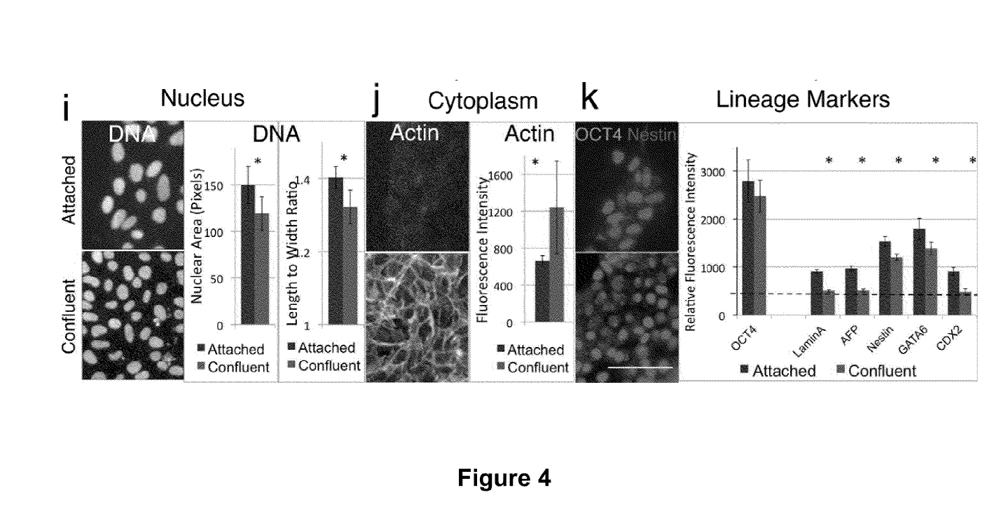

Regarding distinguishing pluripotent from differentiated cells, the results indicate a high rate of successful classification; in colony-wide analysis, 99% of pluripotent windows are identified correctly (see e.g., FIG. 2). The success was validated using established immunocytochemical lineage markers and by confirmation with experienced microscopists. Also presented herein are methods of distinguishing many classes of differentiated cells. For example, ten different classes of differentiated and pluripotent cells were distinguished from each other with 95% confidence. Biological validation, classification performance and statistical analysis are also provided (FIG. 3). Further, a temporal dimension was added and applied to our method to monitoring a process for maintaining pluripotency. The method permits the non-invasive, dynamic evaluation of cell growth and colony maturation while preserving the colony for use. In addition, in textural changes can report structural changes at the molecular level and reflect functional changes of markers of differentiated lineages (see e.g., FIG. 4).

### B. Materials and Methods

1. Cell Growth and Differentiation

Pluripotent hESCs, line H7, were grown feeder-free on Geltrex-coated plates (Invitrogen) and maintained in StemPro (Invitrogen), a defined pluripotency media. Media was changed every other day and colonies were passaged weekly with Collagenase Type IV. Specific and selective differentiation to an epithelial cell type was achieved by culturing for four days in EMIM basal media without bFGF but with the addition of BMP-4 (100 ng/mL) to the media (Erb T M, et al. Stem Cells Dev 2011 Mar. 17; [Epub ahead of print].). Alternately, pluripotent hESCs, lines H7 and HSF6, were grown on mitomycin-treated mouse embryonic fibroblasts and maintained in Knockout DMEM supplemented with 20% Knockout serum replacement, 2 mM L-glutamine, Non-Essential Amino Acids, 100 D/ml Penicillin, 100 flg/ml Streptomycin, 4 ng/ml bFGF (all from Invitrogen). iPSCs were maintained in mTeSR on matrigel (BD Biosciences) according to the manufacturer's recommendations (StemCell Technologies). Mouse embryonic stem cells (mESCs), line R1/E L129 (ATCC line SCRC-1036) were cultured on mouse embryonic fibroblasts (MEFs, Globalstem or Chemicon) maintained in DMEM, with 10% FBS, 1% Pen/Strep and 1% L-Glutamine. Media was changed every other day and stem cell colonies were passaged weekly with either a Pasteur pipette or by enzymatic digestion using Collagenase Type IV as recommended by the provider. Neuronal differentiation of hESC(H7 or HSF6) was produced either by reducing the density of feeders (neurectoderm, NE) (Ozolek J A, et al. In: Turksen K, editor. Human Embryonic Stem Cells: Methods and Protocols. Totowa, N.J.: Humana/Springer; 2009; Ozolek J A, et al. Stem Cells Dev. 2007; 16(3):403-12) or by the addition of the BMP4 antagonist noggin in DMEM/FI2 (Invitrogen) base media, N2 100×, B27 50×, L-glutamine 100×, pen/strep 100×, final concentration 100 ng/ml noggin on Geltrex under feeder free conditions (Chambers S M, et al. Nature Biotechnology 2009; 27(3):275-80; Gerrard L, et al. Stem Cells 2005; 23(9):1234-41; Nakahara M, et al. Cloning Stem Cells 2009; 11(1):5-18). Neural stem cells (NSC) were favored by initial plating of hESC, line H7, in large colonies (>1000 cells/colony); neurectoderm (NE) was favored by initial plating in small colonies (<100 cells/colony); and oligodendrocyte precursors (OPC) were favored by initial plating of single cells passaged by accutase digestion. HEK293 (ATCC) were maintained in DMEM with 2.0 mM L-Glutamine, 100 U/ml Penicillin, 100 μg/ml Streptomycin, and 15% FBS.

2. Imaging

Colonies were imaged via phase-contrast microscopy using a 4× Nikon 0.13 NA objective on a TMS cell culture microscope with a 10M pixel Nikon D40× SLR camera. Phase contrast and Hoffman interference contrast were particularly useful, while brightfield images had lower contrast. Different resolution optics and detectors were evaluated, and low resolution, wide field optics and high resolution cameras produced were found to produce the best classification performance. 20× and 40× objectives provide information on subcellular details, while 4× and 10× objectives emphasize multicellular patterns. The 4× phase contrast objective was suitable because it emphasized features at the scale of whole cells (2.3 mm resolution for green light) and had a field of view sufficient for very large colonies. A 10 M pixel camera with 2.5× coupler lens provided 1 pixel/μm resolution, better than twice the optical resolution, and exceeding the Nyquist sampling frequency. Lower pixel densities did not provide desirable detail for 50-cell colonies. RGB images were reduced to grayscale by selecting the green channel to reduce chromatic aberration and avoid color registration errors in the camera. Intensity line profiles of live cells (FIG. 1) were measured with Image M.

3. Immunostaining

hESC colonies were fixed with 2% paraformaldehyde in PBS buffer, permeabilized with 1% Triton X-100 (Sigma, St. Louis Mo.), and non-specific antibody binding blocked with 10% goat serum. Primary antibodies were diluted in 1% goat serum, spun briefly, and either incubated at 37° C. for thirty minutes or incubated overnight at 0° C. After a wash in PBS-Tween 0.05%, a species-specific fluorescent secondary antibody was added for 60 minutes at 37° C. and then incubated in DNA dye Hoechst 33342 (1:10,000).

As shown in FIG. 3, colonies were immunostained with anti OCT4 (R&D systems) or CDX2 (Biogenex), FOXD3 (Cell Signaling), S0X2 (EMD Chemicals), PDGFR (Santa Cruz), OTX2 (Abeam), nestin (Abeam), and βIII-Tubulin (Covance). Cells were imaged using a Zeiss 10× or 20× objective and Axiocam MR5 camera. The Hoechst image of four independent colonies was segmented by a watershed segmentation, thresholding, and size exclusion using the McMaster Biophotonics Facility Image J plug-ins for Nuclear Counting (Particle Analysis). Cell area in a cropped confluent monolayer was determined by dividing image area by the nuclear count. All images were imported into Adobe Photoshop for final image composition and contrast adjustment. Comparable images were adjusted using constant contrast to allow comparison of channel intensity.

Colonies were immunostained for DNA, OCT4, CDX2, nestin, GATA6 (Abeam), AFP (Santa Cruz), F-Actin (Rhodamine-Phalloidin, Invitrogen) (FIG. 3). Cells were plated in 96 well plates and fixed on day 1 and day 5 after plating when cells became confluent. Plates were scanned using the Cellomics Arrayscan (ThermoFisher). Nuclear intensity was recorded for all markers except for actin, where a cytoplasmic ring was measured. Each measurement was made on 3-20 independent samples totally 1000-5000 cells for high content screening. Nuclear Shape including area and aspect ratio were measured using the Compartmental Analysis Bioapplication.

4. Time Lapse

For time-lapse observation, the microscope and camera were placed in a sterile PCR hood to reduce the risk of contamination. For time-lapse observation, pluripotent hESC were plated in 6 well plates and fed daily with StemPro media. One day after plating, multiple colonies per well were marked with an objective ink marker on the bottom of the plate. Media was replaced with warm Hanks Balanced Salt Solution (HBSS) filled to the top of the well to flatten the meniscus and reduce curvature artifacts in the phase contrast images. HBSS was replaced with media and plates were returned to the CO₂ incubator. Cultures were imaged daily until confluency on day 9.

5. Texture Classification

hESC textures were classified according to a three-stage wavelet-based statistical method developed for Content-Based Image Retrieval (CBIR) (Do M N et al., IEEE Trans Image Processing 2002; 11(2):146-58.). See FIG. 2a.

In the first stage, wavelet decomposition (see e.g., Mallat S G. IEEE Trans Pattern Analysis and Machine Intelligence 1989; 11(7):674-93) was applied to the grayscale of a texturally homogeneous image patch. As wavelet analysis decomposes a signal locally according to orientation and scale, it was especially apt for modeling texture, characterized by intensity randomness at multiple scales. More specifically, an n-level decomposition yielded three detail subbands per level, one oriented horizontally, one vertically, and one diagonally. For this analysis, the absence of textural information in the approximation subband was assumed.

In the second stage, a dissimilarity measure was derived between textural patches from a statistical model of the empirical probability density function (pdf) of the coefficients in the 3n detail subbands, which were assumed to be statistically independent. This assumption, even if an idealization, simplified computation without sacrificing classification performance. These estimated density functions constituted a set of textural features. In the third stage, the statistical dissimilarity measure was used to classify or cluster texture patches. These textural features may be input into a classifier either alone or as part of an ensemble of features, textural and non-textural (cf. Rodenacker K, et al. Analytical Cellular Pathology 2003; 25:1-36). Previous examples include adding border crispness using a Support Vector Machine (SVM) (Jeffreys C G. [Thesis (S.M.)]. Cambridge, Mass.: Massachusetts Institute of Technology; 2004; Mangoubi R, et al. Proc IEEE Intl Symp Biomedical Imaging; 2007) and color, nuclear shape and orientation, etc. using neural networks (Bhagavatula R, et al. Proc IEEE Intl Symp Biomedical Imaging; 2010). As discussed below, the k-nearest neighbor methodology is used for assigning a colony or colony window to a class.

To compute the dissimilarity between textural patches, a pdf estimator was selected to apply to each of the 3n detail subbands. Dissimilarity was quantified using the information divergence (i.e. Kullback-Leibler divergence or KLD). While other divergence measures exist, such as the L1 divergence (∫|f₁−f₂|), Bhattacharyya distance and Renyi Distances, the information divergence is convenient as it admits tractable closed-form solutions for two of the pdf models. For two pdfs f and g, the information divergence is defined:

$${D}_{KL} ( f , g ) = {\int }_{- ∞}^{∞} f ( x ) log \frac{f ( x )}{g ( x )} d x \qquad Equation 1$$

For textures with n decomposition levels, the total dissimilarity between two models is:

$$KLD ( \{ f \} , \{ g \} ) = {\sum }_{i = 1}^{3 n} {k}_{i} ( {D}_{KL} ( {f}_{i} , {g}_{i} ) + {D}_{KL} ( {g}_{i} , {f}_{i} ) ) \qquad Equation 2$$

Generally, so both are added in order to symmetrize the distance between the joint probability density functions of different cell colonies or nuclei. Likewise, {k} are simply weights assigned to particular subbands i. In general, all kᵢ=1 are set, but they might be used to emphasize or penalize certain bands according to prior knowledge.

A variety of different methods may be used to estimate the pdf of the wavelet coefficients and their corresponding KLDs; four are given below. In practice, model selection involves trade-offs between computational simplicity. Several models and the generalized Gaussian (GG) probability density function have been tried and are described below. A comparison against another model is also provided.

It was noted early on (Mallat, IEEE Trans Pattern Analysis and Machine Intelligence 1989; 11(7):674-93) that the pdf of the detail coefficients often resembled the symmetric, unimodal generalized Gaussian distribution (GGD):

$$f ( x ; α , β ) = \frac{β}{2 αΓ \frac{1}{β}} {e}^{- {( \vert x \vert / α )}^{β}} \qquad Equation 3$$

Here, x is the random variable (detail coefficient) and α and β are the width factor and shape parameter, respectively, while Γ indicates the gamma function. The location parameter (i.e. process mean) is assumed to be zero.

The GGD may be used to model a wide variety of symmetric, unimodal density functions. Indeed, special cases include the Gaussian (α=, β=2), Laplacian (α=σ, β=1, and uniform (β→∞) densities for standard deviation σ. The standard deviation of a GGD process is:

$$σ = {α ( \frac{Γ ( 3 / β )}{Γ ( 1 / β )} )}^{1 / 2} \qquad Equation 4$$

The utility of this density function in texture characterization was shown in (Do M N, et al. IEEE Trans Image Processing 2002; 11(2):146-58; Van de Wouwer G, et al. IEEE Trans Image Proc 1999; 8(4):592-8.), who respectively developed moment-matching and maximum likelihood procedures for calculating α and β. This function has been applied to stem cell classification in (Mangoubi R, et al. Proc IEEE Intl Symp Biomedical Imaging; 2008; Mangoubi R, et al. Proc IEEE Intl Symp Biomedical Imaging; 2007).

One of the advantages of this method is that a closed-form solution exists for the 5 KLD between two GGD processes (Do M N, et al. IEEE Trans Image Processing 2002; 11(2):146-58), simplifying computation:

$${D}_{GGD} ( {f}_{1} ∥ {f}_{2} ) = log ( \frac{{α}_{2} {β}_{1} Γ ( 1 / {β}_{2} )}{{α}_{1} {β}_{2} Γ ( 1 / {β}_{1} )} ) + {( \frac{{α}_{1}}{{α}_{2}} )}^{β2} \frac{Γ ( ( {β}_{2} + 1 ) / {β}_{1} )}{Γ ( 1 / {β}_{1} )} - \frac{1}{{β}_{1}} \qquad Equation 5$$

As noted earlier, a challenge in colony image classification is to distinguish the fine-grained pluripotent regions from the differentiated “swampland” and both from the exterior. This was accomplished by subdividing images into non-overlapping windows of constant size, each of which is classified independently using the GGD density function to model the statistical variation of the wavelet coefficients. At the scales of interest, these windows will exhibit textural isotropy (i.e. a lack of directionality or an intensity gradient), and hence this model is favored for its simplicity and robustness. As intra-class textural heterogeneity means that not all differentiated, pluripotent, or exterior windows exactly resemble one another, windows were classified by comparison against expert-classified samples in a model library compiled from four images (three pluripotent, one differentiated) that serve as a training or learning set. The library is illustrated below.

### C. Results

1. Summary

The non-invasive imaging methodology were tested and chemically validated on three different data collections, confirming that texture analysis can indeed be a non-invasive biomarker. First, the methodology recognizes pluripotent from differentiated (e.g., trophectoderm), cell colonies (FIG. 1).

Second, the classification process was expanded to ten cell classes (FIG. 3 a-j) with validation (FIG. 3k-r), demonstrating that texture analysis can distinguish between these classes without staining (FIG. 3 s-w). Further statistical analysis are also presented (FIG. 3x-z), and includes implications for untrained clustering. In addition, it is shown that non-invasive texture analysis can be used to monitor protocols for maintaining and enhancing pluripotency (FIG. 4 a-h). Chemical validation is also provided (FIG. 4 i-k).

2. Colony Morphology in Pluripotent Vs. Differentiated Stem Cells

Human ESC colonies were maintained in the pluripotent state (FIGS. 1A, C, J) as confirmed by immunostaining for molecular markers of pluripotency (OCT4, FIG. 1C). Differentiated cells (FIG. 1b, e, h) were produced by treatment with the growth factor bone morphogenic protein BMP4 to form trophectoderm (TE) as confirmed by immunostaining (CDX2, FIG. 1e) (Erb T M, et al. Stem Cells Dev 2011 Mar. 17; [Epub ahead of print]; 2011 September; 20(9):1601-14.). Trophectoderm is a well-spread epithelial monolayer. These two validated developmental stages were used to develop a textural approach to colony morphology.

Stem cell colonies change morphology during differentiation in part due to a 40% increase in nuclear and cellular diameter (FIGS. 1E and F) and also because of an increase in heterogeneity of cell size since the standard deviation is 80% and 50% larger for differentiated cells and nuclei, respectively (FIGS. 1E and F). Compare the colonies in FIGS. 1J and 1K. Pluripotent colonies (FIG. 1J) exhibit a fine-grained, homogeneous texture within a crisp, clearly defined border around each cell. The well-defined cells are of uniform size, some with white borders (a phase halo artifact due to a lens effect of domed cells). In contrast, differentiated colonies (FIG. 1K) have dark centers, irregular, thin cell edges (no white lines). Each cell has a “fried egg” appearance. Horizontal line scans (FIGS. 1L and 1M) show that both images exhibit considerable intensity variation over distance, but the two differ by the scale at which random fluctuation occurs; grayscale levels in the fine-grained pluripotent colony fluctuates at a comparatively higher spatial frequency (FIGS. 1L and 1M). The intensity profile depends not only on cell and nuclear diameter (x and y dimensions), but also on whether cell edges are abrupt or graded (physical profile in the z dimension).

This spatial frequency contains composite information regarding the edges in an image and is an integrated measure of the size, number, and shape of the cells in the image. Small, compact cells have more sharp edges than large cells with gradually graded cell-cell borders. Stem cell image texture was classified by quantifying scale-dependent statistical variation using multiresolution wavelet analysis, which decomposes a signal locally at various scales. As the multiscale decomposition is statistical in nature, sample coefficients of these decompositions have different histograms, depending on whether the colony is pluripotent or differentiated, as FIGS. 1G, 1H, and 1I show. In FIGS. 1G and 1H, the coefficients in the second and third detail subbands, which account for spatial variation at scales of 2² and 2³ pixels, respectively. Thus, the coefficients from these subbands of the pluripotent colony have a probability density function with a noticeably wider center and thicker tails, indicating more numerous edges at small scale and hence more and smaller cells. In FIG. 1I, the situation is reversed at the sixth detail subband (scale 2⁶ pixels), indicating that the differentiated image contains more widely spaced edges and hence larger cells. The scale most sensitive to stem cell differentiation is similar to the average diameter of pluripotent and differentiated stem cells, or 69±30 pixels and 82±40 pixels, respectively. To be fully descriptive for a colony image, this multiscale statistical description was generalized to two dimensions, as described below, below and exploit it to classify stem cells.

3. Colony Library and Hierarchical Windowing

The flow diagram in FIG. 2a illustrates the comparison of an untested image against a reference library of images that is validated by independent means. In some implementations, accurate identification of unknown samples depends on correct validation of classes in the library and sufficient coverage of candidate cell types with a comprehensive library. Wavelet Decomposition provides the PDF at various subbands and the distance or dissimilarity between all pairs of images is estimated by the KLD. Statistical comparison among images provides the criteria for grouping of images into distinct classes. The classes in the library are sorted in FIG. 2b. Each square entry in the matrix represents the KLD between a pair of sample windows from the learning set, with the color of an entry indicating the degree of dissimilarity (see scale). The intra-library KLD shows the grouping of library textures into six classes, one differentiated, three exterior, and two pluripotent (from top left to bottom right), shown as bluish (low dissimilarity) blocks clustered along the diagonal of the matrix of KLD between pairs of colonies in the library. Note the resemblance, though the two remain distinguishable, between the first (differentiated) and last (pluripotent) classes, shown in the greenish bar off-diagonal in the top left corner, and the textural heterogeneity of the differentiated class, with its lighter shade of blue.

Window class assignment uses a k-Nearest Neighbor classifier. After computing the KLD dissimilarity between the unknown window to each library model, the k models with the smallest dissimilarity (i.e. nearest neighbors) “vote,” and the class with the most “votes” wins. To reject ambiguous cases, models are only allowed to “vote” if their dissimilarity is below some ceiling value, and the end result is only accepted if at least kₙ≦k “votes” concur. In practice, classification is shown to be robust to the choice of k, kₙ, and the dissimilarity ceiling.

For ease and speed of computation, windows are chosen to be fairly large (256×256 pixels). Blue windows are classified as pluripotent, green as differentiated, and red as exterior. Gold windows are unknown or could not be classified. These are very few in number. Several windows contain heterogeneous mixtures of textural classes, e.g. pluripotent and exterior. Such windows represent the superposition of two (or more) pdfs. To address this issue, classification of mixed windows was refined via hierarchical image windowing (FIG. 2c). After the classifier identifies these windows using spatial reasoning rules, they were quartered and re-classify in the same manner as above. This process may be reiterated until characteristic features (e.g. differentiated cellular clumps, pluripotent cells separated by white lines, etc.) are on the order of the window size. Minimum scale in this application was thus 64×64 pixels. This iterative process provides segmentation of heterogeneous images that contain multiple cell types or colony borders.

Two spatial reasoning rules identify textural border windows for refined classification. First, select windows failing the kNN test. Second, select any differentiated window that borders upon a window of another class; since differentiated hESC regions are characterized by a type of textural inhomogeneity, border windows are prone to being misidentified as differentiated. The input and results of a segmentation of a differentiated colony are shown in FIG. 2d. In this example, definition of the colony border is accurate to the smallest window (64×64), the size of individual cells and is a corollary benefit of texture analysis by hierarchical windowing.

4. Pluripotent vs. Differentiated Colonies: Classification Performance

Classification of inhomogeneous hESC colony images was quite successful. Typically, identification of pluripotent windows was near perfect (99%, 90% confidence interval (CI) [0.9812, 0.9986]). All but one window among the very small percentage of misclassified pluripotent windows were assigned to the colony exterior, so pluripotent windows were essentially never classified as differentiated. This could be significant for tissue engineering application in which a surgeon aims to transplant essentially no pluripotent cells into patients.

Differentiated and exterior accuracy was 89% (90% CI [0.8547, 0.9196]) and 96% (90% CI [0.9432, 0.9812]), respectively. Usually, hESC colonies spread out as they differentiate, and thus approximately 70% of misclassified differentiated windows were assigned to the exterior with the remainder being unclassifiable. Thus no differentiated cell was misclassified as pluripotent. This could be significant for large-scale hESC cultivation in which the goal is to produce pluripotent colonies that are uncontaminated.

Results in FIG. 2e give accuracy results for a variety of classification (k, kn) and modeling (wavelet basis) parameters. Blue indicates pluripotent, green differentiated, and red extracellular substrate. The bars indicate the 90% CI and the diamond the mean of the distribution. Note that pluripotent accuracy was highly robust to changes in these values. Differentiated accuracy varies but was acceptable with wavelets of eight taps or less (Sym4 and left).

As noted, these results used GGD densities to model wavelet coefficients. By way of comparison, results with SuS models are shown in FIG. 2f for the “typical” set of model parameters. While pluripotent accuracy is relatively unchanged, differentiated accuracy decreased noticeably (82%, down from 89%), validating the use of GGD density models in this application.

The non-invasive algorithm's results were validated by consulting a microscopist's evaluation of the same phase contrast images that were directly analyzed by computer vision, as well as by immunochemical validation of samples of cells grown under identical conditions. Results of the application of the algorithm to statistically classify ten different categories of stem cells, non-invasively, is described below.

5. Non-Invasive Classification of Multiple Types of Differentiated and Pluripotent Stem Cells with Biological and Statistical Validation

The classification was expanded from two classes to ten classes. Biological and statistical validation were also performed. Briefly, a variety of cell types were purchased or derived from pluripotent stem cells and characterized by developmental immunomarkers. Phase contrast images of living samples of the various lineages were characterized by texture analysis, were distinguished statistically, and were correctly resorted based solely on texture.

a. Distinguishing Pluripotent vs. Multipotent vs. Differentiated Cells Using Immunomarkers

In FIG. 3, representative images of pluripotent, multipotent, and differentiated cells are shown. FIGS. 3a-j show enlarged phase contrast images of the following ten live cell classes: 1) pluripotent hESCs line H7 in the feeder-free media, StemPro (H7sp, FIG. 3a); 2) iPSCs, line IMR90 in the feeder-free media, mTeSR (IMR90, FIG. 3b); 3) hESCs on feeders (H7fdr, FIG. 3c), 4) mESCs, line RUE on feeders (RIE, 5d); 5) neural stem cells differentiated from hESCs in noggin containing media (NSC, FIG. 3e); 6) human embryonic kidney cells, line HEK293 (HEK, FIG. 3f); 7) trophectoderm differentiated from hESC using BMP4 (TE, FIG. 3g); 8) neurectoderm differentiated from hESC using noggin (NE, 5h); 9) mouse embryonic fibroblasts from day 14.5 embryos (MEF, FIG. 3i); and 10) oligodendrocyte precursor cells differentiated from hESC using noggin (OPC, FIG. 3j). Larger windows cropped from images of each class of cells, five to thirty windows per class, were used to evaluate similarities among cell types.

FIGS. 3k-r confirmed stem cells and differentiated derivatives via immunostaining for markers of each lineage. Specifically, hESC pluripotency was confirmed by immunostaining with markers OCT4 and FOXD3 (FIG. 3k) and SOX2 (FIG. 3l) (Fong H, et al. Stem Cells 2008; 26(8):1931-8; Liang J, et al. Nat Cell Bioi 2008; 10(6):731-9). NSCs were confirmed with βIII-Tubulin and low nestin expression (FIG. 3m) (Barberi T, et al. Nature Biotechnology 2003; 21(10):1200-7.) and OTX2 (FIG. 3n). OPCs were confirmed with the presence of PDGFR (FIG. 3o) and nestin (FIG. 3p) and by the absence of βIII-Tubulin (not shown) (Rao R C, et al. Stem Cells 2009; 27(1):116-25.). NE was confirmed with the presence of nestin (FIG. 3q) and the absence of PDGFR and βIII-Tubulin (not shown) (Shin S, et al. Stem Cells 2006; 24(1):125-38.), and TE was confirmed with CDX2 (FIG. 3r) (Erb T M, et al. Stem Cells Dev 2011 Mar. 17; [Epub ahead of print].).

The ability of the texture analysis algorithm to distinguish and accurately classify various types of colonies was examined. In FIGS. 3s-z, provides observations and insights on the ability of the algorithm to statistically distinguish between colonies within and across classes, in order to distinguish between classes of colonies, and to classify and cluster colonies into classes. As explained below, FIGS. 3s-w compare colonies within and across classes, while FIGS. 3x-z provides a summary of aggregate comparisons at the class level, including statistical test results at the 0.05 significance level. Additionally, results from an algorithm for clustering each colony with sister colonies in the same class are described.

Textural analysis was performed by first manually identifying and cropping homogeneous textural windows within images. Subsequently, a five-level multiresolution wavelet decomposition was applied to these windows using the Daubechies-4 wavelet. Features were estimated by modeling each subband using the generalized Gaussian density (GGD) as described above.

b. Distinguishing Pluripotent vs. Multipotent vs. Differentiated Cells Using Non-Invasive Texture Image Analysis

In FIG. 3s, the ten cell colony types described in FIGS. 3a-j were examined. As before, color indicates the value of the KLD between a pair of windows, with the color scale shown on the right. Blocks of entries along the diagonal, outlined in white, show the KLD dissimilarity for the elements in a given class; classes are presented in the same order as FIGS. 3a-j. Specifically, from top left to bottom right: H7sp, IMR90, H7fdr, RIE, NSC, HEK, TE, NE, MEF, and OPe. The diagonal entries that compare a colony window to itself are dark blue, indicating a KLD of zero (no self-dissimilarity, i.e. positive control). For pairs of windows in the same class, the corresponding entries are close to blue, indicating small intra-class KLD relative to the off-diagonal blocks that tend towards red and correspondingly larger inter-class KLD. Based on this figure, it is evident that human embryonic or induced pluripotent stem cells can be distinguished from differentiated human cells and mouse cells.

In FIG. 3t, the three pluripotent human cell classes, H7sp (hESC), IMR90 (iPSCs), H7fdr (hESCs), and the pluripotent mouse class RIE (mESCs), whose images are shown in FIGS. 3a-d were examined. Only two of the four classes, H7sp and IMR90, both grown in feeder-free conditions, were essentially indistinguishable.

A comparison of H7sp and H7fdr, shown in FIGS. 3a and 3c illustrates that morphologically, stem cells on feeders differ in terms of cell-cell contacts at cell edges. This is illustrated by the white spaces between cells (phase halos, FIG. 3c), although nuclear and cell size are all similar. Wavelet textural analysis is sensitive to intrinsic cellular features, as was verified statistically in FIGS. 3x-z and explained below.

RIE is shown in FIG. 3d. In FIG. 3t, these cells are distinguishable from the other three pluripotent cell classes due in large part to their smaller average nuclear size (5 flm vs. 8 flm).

In FIG. 3u, the following multipotent stem cell and somatic cell colonies were compared: NSC, HEK, TE, NE, MEF, and OPC (shown in FIGS. 3d-j). The figure shows that colony pairs from different classes are distinguishable: nearly all off-diagonal blocks tend to red, indicating large inter-class dissimilarity. For instance, ectodermal cells (TE and NE, shown in FIGS. 3g-h, respectively) that form flat epithelial like sheets may be distinguished from each other. Note that the KLD dissimilarity between NE and MEF colonies, shown in FIGS. 3h-i, respectively, is not as large as the inter-class dissimilarity between other class pairs, due to their similar size and elongated, fusiform shape. Nevertheless, these two classes are still statistically distinguishable, as explained below and in FIGS. 3x-z. Finally, OPCs are distinguishable from other classes because they are well spread with a convex periphery and do not form cell-cell junctions, while NSCs are also distinguishable because the cells in the colony are tightly knit.

FIG. 3v shows a comparison of mouse cells: mESC colonies RIE (FIG. 3d) with MEF (FIG. 3f). The dark red of the off-diagonal blocks shows that the two classes may easily be distinguished, while the deep blue of the MEF indicates low intra-class KLD and hence high textural homogeneity. In contrast, the varied color pattern in the block of mESC colonies indicates high intra-class textural variation.

FIG. 3w shows in detail two colonies with very similar visual appearance, RIE and NSC (FIGS. 3d-e, respectively). Despite resemblance between these colonies by eye, wavelet-based texture analysis easily distinguishes the two.

The last item in FIG. 3w, originally believed to be NSC, does not belong to either RIE or NSC (or, in fact, any of other eight classes). Upon further examination of the corresponding image, it was discovered to have been poorly cropped, with half the image consisting of exterior, and the other consisting of the colony cells. Upon properly re-cropping, the texture analysis algorithm confirmed (not shown) that this colony indeed belongs to the NSC class. This result shows that the algorithm can successfully detect an outlier or an anomalous cropping (negative control) missed by human observation.

c. Further Statistical Validation of Non-Invasive Texture Analysis

FIG. 3x provides pairwise KLDs from ten representative colonies, one selected from each class. Each representative was chosen as the colony with the smallest average intra-class dissimilarity, and the degree to which pairs of representatives are distinguishable can be seen from the color patterns.

FIG. 3y presents the statistics of the non-parametric, two-sample Kolmogorov-Smirnov (KS) test between pairs of classes, using all available items from each class. This test provides pairwise comparison between the distribution of the populations of intra-class and inter-class KLDs. For instance, the entry in row 2, column 6 shows the two-sample KS statistic comparing the intra-class KLDs within IMR90 to the inter-class KLD dissimilarity between items in IMR90 and items in HEK. Unlike the previous figures, this plot is not symmetric. For example, row 6, column 2, shows the intra-class dissimilarity within HEK (rather than IMR90, as in row 2, column 6) and the inter-class divergence from IMR90 to HEK (as in row 2, column 6).

In FIG. 3z, the results of these KS tests, based on statistics from FIG. 3y, are shown for the 0.05 significance level. The null hypothesis is that the intra-class and inter-class KLD dissimilarities are from the same population, or equal in probability distribution, while the alternate hypothesis states that the inter-class KLDs are larger than the intra-class KLD dissimilarities and have a different probability distribution. A black entry in FIG. 3z means that the null hypothesis is accepted, meaning that the two populations come from the same distribution, while a white entry means that the alternate hypothesis is selected, indicating that the inter-class KLD dissimilarity is larger and the two classes are distinguishable. Note that the row indicates which intra-class dissimilarity is being tested, while the row and column indicate which the selected inter-class dissimilarity. The diagonal blocks are black as the null hypothesis holds that each class is similar to itself.

For all other tests but one, the alternate hypothesis is accepted indicating that the corresponding class pair is distinguishable. The sole exception indicated the difficulty in distinguishing between the intra-class dissimilarity of IMR90 and the inter-class H7sp-IMR90 dissimilarity.

The two sample t-statistics for the mean KLD dissimilarity yielded the same decisions as the two sample KS test in FIG. 3z (data not shown).

d. Clustering of Similar Stem Cell Colonies Together

Colonies were grouped based on the KLD dissimilarities between them. The kNN classifier discussed above with k=5 was applied. Treating the three pluripotent cell types (H7sp, IMR90, and H7fdr) as a single class, all but four of the 133 image windows in our data set were successfully clustered. Variations on this rule yielded similar performance.

6. Time-Lapse Analysis of Pluripotent Colony Phenotype

One of the strengths of non-invasive image analysis is the ability to measure dynamic spatiotemporal changes of the same group of cells over time while preserving their usefulness. Thus, if the approach is validated, the need for staining to confirm a protocol's ability to produce the desired change would be reduced. This example demonstrates that non-invasive texture analysis can be used to monitor pluripotent colonies from early attachment to colony compaction as cells approach confluency in StemPro media, changed daily.

Phenotype of compacting pluripotent stem cells is characterized by three complementary measures: first, by live-cell non-invasive image texture analysis, second, by structural measurements (nuclear size and cytoskeletal organization) and third, by functional measurements (developmental markers). Thus the structural and functional measurements validate the noninvasive texture analysis.

a. Non-Invasive Texture Analysis During Colony Compaction

A protocol intended to maintain pluripotency was applied to each of six independent colonies for 9 days. One of these colonies is shown in FIGS. 4a-e on days 1, 3, 5, 7, and 9. The objective was to obtain a colony as in FIG. 4e with small cells with tight junctions within a uniform colony. The media was refreshed daily and texture was evaluated just before media changes. Sample colonies were immunostained after attachment and upon compaction and lineage markers were evaluated on these colonies.

FIG. 4f shows, for each of the six colonies, The KL divergence between each of days 1 to 8, and day 9 of a reference colony that was fully compacted and validated immunocytochemically. From day 1 to 9, by day 7 the KL divergence approaches zero, meaning that all the colonies' textural features resemble those of the target colony. This was verified visually by microscopists for each of the colonies.

To evaluate the similarities among colonies, the KL divergences of each of the six colonies on each of the 9 days was compared to all other colonies on all other days (FIG. 4g), for a total of 54 comparisons. The order of presentation clusters all colonies for day 1 followed by all colonies for day 2 and so on. The table indicates incipient textural changes on day 4. FIG. 4h shows the results of the Kolmogorov Smirnov test that compares the population intra-day KL divergences vs. inter-day KL divergences. A black entry indicates that the KL distance populations for two days are the same at the 5% significance level, while a white entry indicates they are different. The textures of pluripotent colonies for days 1-2 are essentially indistinguishable (black entries), while subsequent changes are distinguishable at the 5% significance level every two to three days.

b. Structural Changes During Colony Compaction

To validate whether the textural changes of pluripotent colonies reflect changes in cellular structure, high content screening to evaluate pluripotent colonies was used, a day after attachment vs. confluency in separate, fixed 96 well plates. Nuclear and cell area contributed to different subbands during trophectoderm differentiation (FIG. 1) so nuclear and cytoplasmic morphology was evaluated using Hoechst DNA dye for nuclei and rhodamine-phalloidin for filamentous actin at the periphery of the cytoplasm. Attached colonies had larger, more elongated nuclei than confluent colonies as measured by the cross sectional area and length to width ratio (FIG. 4i). The cytoplasm of small, recently attached colonies contained reduced amounts of disorganized actin filaments while confluent colonies contained bright, peripheral actin bands, characteristic of columnar epithelium with established cell-cell junctions (FIG. 4j). The rounded apical surface that produces the phase halo in small colonies becomes flat-topped in taller, columnar epithelium, thus changing colony texture at the larger subbands. Therefore, the textural measurements shown in FIGS. 4f thru 4h reflect the structural changes associated with colony compaction and thus texture can be used to monitor subtle changes in cell shape.

c. Functional Changes During Colony Compaction

To further evaluate whether the textural changes reflect functional changes during pluripotent colony compaction, molecular markers of pluripotency and early differentiated lineages were measured by high content screening. Overall, pluripotency markers did not change upon confluency. The marker OCT4 (green, FIG. 4k) decreased slightly, but not significantly (2791±441 to 2577±331 P>0.07, 10 wells, 2000 cells each condition, graph, FIG. 4k). No changes were observed in staining of smaller numbers of cells for the pluripotency markers, SOX2, REX1, HNF3b, while decreases in Nanog, FGF5, and an increase in FoxD3 were not statistically significant (not shown). In contrast, differentiation markers for endoderm (AFP), neurectoderm (nestin, red), extraembryonic endoderm (GATA6) and trophectoderm (CDX2) were positive after attachment, but decreased during growth to confluency (FIG. 4k, >3 samples, >1000 cells). Lamin AIC, typically absent from pluripotent cells (Constantinescu D, et al. Stem Cells 2006; 24(1): 177-185), was present in the nuclear envelope after attachment but was reduced to background intensities upon colony growth to confluency (FIG. 4k). Typically, the expression of differentiated lineage markers in pluripotent cells is understood as a commitment to further differentiation (Hough S R, et al. MF. PLoS ONE 2009; 4(11):e7708; Toyooka Y, et al. Development 2008; 135(5):909-18.). However, differentiated marker expression was transient, and colonies cultured under these conditions routinely remained pluripotent for up to 40 passages (from 30 to 70). Therefore, expression of differentiation markers does not reflect a commitment to differentiate.

Co-expression of pluripotent and differentiation markers is not mutually exclusive and might have functional significance, indicating a priming for selective fate decisions without a commitment to continue differentiation (Hong S-H, et al. Cell Stem Cell 2011; 9(1):24-36). Different sub-states of pluripotency have been observed with preferred lineage competence (Stewart M H, et al. Nat Meth 2010; 7(11):917-922; Narsinh K H, et al. J Clin Invest 2011; King F W, et al. Stem Cells and Development 2009; 18(10):1441-1450). The present results demonstrate that expression of lineage markers occurs dynamically and reversibly early after passage in colonies that are texturally distinguishable from the final compacted state. The developmental potential of these two states are different. Pluripotent cells that were passaged as small colonies or single cells into neural differentiation media formed oligodendrocyte precursor cells (FIG. 3 j, 0, and p), while pluripotent cells that were passaged as very large compacted colonies produced neural restricted stem cells (FIG. 3 e, m, n).

### Example 2

### A Unified Approach to Expectation-Maximization and Level Set Segmentation Applied to Stem Cell and Brain MRI Images

### A. Introduction

In this example, a unified approach to Expectation-Maximization (EM) and Level Set image segmentation is provided that combines the advantages of the two algorithms via a geometric prior that encourages local classification similarity. Compared to level sets, the method disclosed herein increases the information returned by providing probabilistic soft decisions, is easily extensible to multiple regions, and does not require solving Partial Differential Equations (PDEs). Relative to the basic mixture model EM, the unified algorithm improves robustness to noise while smoothing class transitions. The versatility and advantages of the algorithm are illustrated on two real-life problems: segmentation of induced pluripotent stem cell (iPSC) colonies in phase contrast microscopic images and information recovery from brain magnetic resonance images (MRI).

The similarity of Expectation-Maximization (EM) and Level Set algorithms is exploited to present a unified segmentation routine combining their advantages. A soft classifier, the algorithm disclosed herein returns the probability of class inclusion and thus yields more information than level sets, which return only hard decision labels. The present approach is different than other stochastic level set-like algorithms, such as K. Pohl et al., IPMI 2007, LNCS 4584, 2007, 26-37 and G. Tsechpenakis et al., IEEE Trans. Image Proc. Vol. 18, No. 10, October 2009, 2316-2329, which respectively utilize mean field theory and conditional random fields. Compared to level sets, the approach disclosed herein eases implementation by requiring no solution to PDEs or maintenance of a level set function and easily extends to multi-region segmentation without tracking multiple level set functions. In comparison to EM, the presently disclosed algorithm greatly enhances noise robustness by using a prior based on the geometry of the soft decision, thereby encouraging local classification similarity in a manner that extends the insight in Zhang Y et al., IEEE Trans. Medical Imaging, 20:1 Jan. 2001, 45-57. The present example demonstrates this approach to the disparate tasks of iPSC colony and brain MRI image segmentation.

### B. Computational Methods

1. Unified EM/Level Set Segmentation

The hidden data is the true classification Z(x), which assigns a class to each pixel x in image 15 domain Ω from class set C. Observations Y(x) are generated stochastically from Z according to parameters θ.

a. EM Segmentation

The EM algorithm A. Dempster et al., J. Royal Stat. Soc., Ser. B (Methodological). Vol. 39, No. 1 (1977) 1-38 is an iterative method for estimating Z from Y that is commonly used to segment biomedical images such as brain MRI W. Wells et al., IEEE Trans. Medical Imaging. Vol. 15, No. 4, August 1996, 429-442. At each iteration k, the algorithm performs an E-Step that updates the soft classification for cεC according to Bayes' Rule:

$${p}_{k + 1} ( Z = c \vert Y ; {Θ}_{k} ) = \frac{{p}_{k} ( Y \vert Z = c ; {Θ}_{k} ) {p}_{k} ( Z = c )}{{\sum }_{l ∈ C}^{ } {p}_{k} ( Y \vert Z = l ; {Θ}_{k} ) {p}_{k} ( Z = l )} \qquad Equation 6$$

Indexing is suppressed by x for legibility. In the M-Step, parameter estimates θ (including prior Pₖ(Z)) are updated via maximum likelihood:

Θ=arg_{θ}maxp(Y|Θ;Z) Equation 7

If pₖCZ) is estimated from the mixture ratio:

$${p}_{k + 1} ( Z = c ) = \frac{1}{\vert Ω \vert} {\sum }_{x ∈ Ω}^{ } {p}_{k} ( Z ( x ) = c \vert Y ( x ) ; {Θ}_{k} ) \qquad Equation 8$$

The result is the finite mixture model EM (FMM-EM).

b. Level Set Image Segmentation

Level set algorithms have enjoyed extensive use in image segmentation for over a decade (e.g. V. Casselles et al., “Geodesic Active Contours.” ICCV 1995, T. Chan et al., IEEE Trans. Image Proc. Vol. 10, No. 2, February 2001, 266-277). The region competition formulation (S. Zhu et al., IEEE Trans. Pattern Analysis and Machine Intelligence. Vol. 18, No. 9, September 1996, 884-900) segments an image into two classes c₀ and c₁by minimizing a functional of an auxiliary surface φ:

$$E ( φ , Θ ; Y ) = - {\int }_{Ω}^{ } H ( φ ( x ) ) \cdot log p ( Y ( x ) ) \vert {c}_{0} ; Θ ) d x - {\int }_{Ω}^{ } H ( - φ ( x ) ) \cdot log p ( Y ( x ) ) \vert {c}_{1} ; Θ ) d x - v {\int }_{Ω}^{ } H ( φ ( x ) ) d x + λ {\int }_{Ω}^{ } ‖ ∇ H ( φ ( x ) ) ‖ d x \qquad Equation 9$$

As the regularized Heaviside step function HO demarcates the two regions (cₒ={x|φ(x)>0}), the zero level set of φ thus partitions the image. Constants ν and λ respectively penalize 15 the area of c₀ and the length of the segmenting contour. This functional is minimized via gradient descent:

$$\frac{\partial φ}{\partial t} = {H}^{′} ( φ ( t ) ) \cdot ( log \frac{p ( Y \vert {c}_{0} ; Θ ( t ) )}{p ( Y \vert {c}_{1} ; Θ ( t ) )} + υ + λ k ( φ ( t ) ) ) \qquad Equation 10$$

where κ(φ) is the curvature of the iso-contours of φ.

c. A Unified Approach

For two classes, we unify these methods by specifying that the level set function φ is equal to the log-likelihood ratio of classification at steady-state:

$$φ = log \frac{p ( Z = {c}_{0} ) \vert Y ; Θ )}{p ( Z = {c}_{1} ) \vert Y ; Θ )} \qquad Equation 11$$

Since p(Z=c₁|Y; θ)=1−p(Z=c₀|Y; θ), this is equivalent to the “log odds” approach of K. Pohl et al. (IPMI 2007, LNCS 4584, 2007, 26-37) and implies that φ relates to the soft decision according to the logistic function p(Z=c₀|Y; θ)=(1+e^{-φ})⁻¹.

The log-likelihood ratio of equation (6) at steady-state is:

$$log \frac{{p}_{k} ( Z = {c}_{0} \vert Y ; {Θ}_{k} )}{1 - {p}_{k} ( Z = {c}_{0} \vert Y ; {Θ}_{k} )} = log \frac{{p}_{k} ( Y \vert Z = {c}_{0} ; {Θ}_{k} )}{{p}_{k} ( Y \vert Z = {c}_{1} ; {Θ}_{k}} + log \frac{{p}_{k} ( Z = {c}_{0} )}{1 - {p}_{k} ( Z = {c}_{0} )} \qquad Equation 12$$

If we assume that parameter estimates θ, and hence posterior Pₖ(Y|Z; θ), are equal for both the EM and level set, we may use relation (11) to solve for the prior Pₖ(Z=cₒ) that causes the right hand side of (10) to be zero:

$${p}_{k + 1} ( Z = {c}_{0} ) = \frac{{p}_{k + 1}^{s} ( Z = {c}_{0} ) {p}_{k + 1}^{m} ( Z = {c}_{0} )}{{\sum }_{l ∈ C}^{ } {p}_{k + 1}^{s} ( Z = l ) {p}_{k + 1}^{m} ( Z = l )} \qquad Equation 13$$

This prior is a function of terms pₖ^{m}(Z) and pₖ^{s}(Z) which are defined below and whose significance is also described.

$$\begin{matrix} {p}_{k + 1}^{m} ( Z = {c}_{0} ) = {( 1 + {e}^{- v} )}^{- 1} & Equation 14 \\ {p}_{k + 1}^{s} ( Z = {c}_{0} ) = {( 1 + {e}^{- v} )}^{- 1} & Equation 15 \\ L = log \frac{{p}_{k} ( Z = {c}_{0} \vert Y ; {Θ}_{k} )}{1 - {p}_{k} ( Z = {c}_{0} \vert Y ; {Θ}_{k} )} + λ k ( {p}_{k} ( Z = {c}_{0} \vert Y ; {Θ}_{k} ) ) & Equation 16\end{matrix}$$

Naturally, pⁱ(Z=C₁)=1−pⁱ(Z=cₒ) for iε{m, s}. Given this prior, steady-state solutions to equations (6) and (10) coincide under the log-likelihood relation (11).

The factorization process in equation (13) represents two distinct aspects of the algorithm. The term p^{m}(Z) is a function of v which is constant with respect to both iteration k and coordinate x; similar to (8), this mixture prior acts as a constant weight on classification. The quantity L is the previous log-likelihood of classification smoothed via flow-by-curvature and varies with respect to both k and x. The term p⁸(Z) biases coordinates towards the classification of their neighbors. The smoothing prior thus creates stochastic dependence in a spatially localized neighborhood to encourage local classification similarity. As both prior terms are derived from the geometry of the previous soft classification Pₖ(Z|Y; θ), the combined (13) is referred to as the geometric prior.

The disclosed unified approach is implemented as an EM. The E-Step is implemented as (6) with priors as in equations (13)-(16), and the M-Step is implemented as (7). In practice, this algorithm is extended by substituting other mixture or smoothing priors. Thus, equation (8) may replace mixture prior (14). Likewise, as flow-by-curvature may be numerically expensive to compute, it may be desirable to replace (15) with some simpler smoothing function, such as convolution with a Gaussian kernel of bandwidth σ:

pₖ₊₁^{s}(Z=c)=G•ₖ(Z=c|Y;Θ) Equation 17

Another alternative is Y. Zhang et al., IEEE Trans. Medical Imaging, Vol. 20, No. 1, January 2001, 45-57, which computes a smoothing prior using Markov Random Fields.

Furthermore, as the EM natively extends to an arbitrary number of classes, the unified approach may be extended to multi-region segmentation without requiring computational machinery to fill gaps or eliminate overlap between regions as in many multiphase level set algorithms.

C. Segmentation of Unstained Induced Pluripotent Stem Cells (iPSC)

1. Introduction

Induced pluripotent stem cells offer many therapeutic and research opportunities, but require constant monitoring to assess quality and pluripotency. The present approaches are: (1) chemical staining, which is rapid and consistent but destructive, rendering a portion of the colony unfit for further experimental or therapeutic use, and (2) visual inspection by a trained microscopist, which is non-invasive but time-consuming and subjective. As an alternative, we have accurately identified stem cells (N. Lowry et al., “Nonparametric Segmentation and Classification of Small Size Irregularly Shaped Stem Cell Nuclei Using Adjustable Windowing” ISBI 2010; R. Mangoubi et al., “Non-Invasive Image Based Support Vector Machine Classification of Human Embryonic Stem Cells” ISBI 2007; R. Mangoubi et al., “Performance Evaluation of Multiresolution Texture Analysis of Stem Cell Chromatin” ISBI 2008) using multiresolution texture analysis as a non-invasive, non-destructive pluripotency biomarker.

A Multiresolution texture analysis accurately classifies homogeneous image textures patches, and we have successfully applied this procedure to both cell nuclei and colonies. Effective segmentation prior to classification is important for automating pluripotency monitoring, and we have successfully done so for individual nuclei. For colonies, this task is more challenging; mean intensity varies little between the colony and its growth media, and the high local variability of multiresolution texture features often traps segmentation routines in undesirable local minima.

As described below, the segmentation of a set of iPSe colony images is automated by combining our unified EM/level set algorithm with a wavelet energy-based texture feature.

2. Methods

a. Data Collection

Induced pluripotent stem cells (iPSCs), line ESIMR90-3 (a gift from J. Thomson, U. Wisconsin) were cultured in feeder-free mTeSR media (Stem Cell Technologies) on Matrigel coated plates (BD Sciences). Colonies were imaged 1-3 days after passaging with collagenase (Invitrogen) via phase-contrast microscopy using a 4× Nikon 0.13 NA objective on a TMS cell culture microscope with a 10M Pixel Nikon D40× SLR camera. Light levels and exposure were set to minimize noise while avoiding detector saturation. Images were reduced to grayscale from the green channel as phase contrast optics are optimized for green light and the camera's Bayer color filter produces slight color registration errors.

b. Texture Features

Segmentation was performed using texture features based on wavelet energy, which were also used to classify hESC colony images (R. Mangoubi et al., “Non-Invasive Image Based Support Vector Machine Classification of Human Embryonic Stem Cells.” ISBI 2007). At iteration k, conditional probabilities were computed as follows:

$$log {p}_{k} ( Y ( x ) \vert Z = c ; {Θ}_{k} ) α - {\sum }_{b ∈ \{ h , v , d \}}^{ } {( {r}^{b} ( x ) - {μ}_{k}^{b} ( c ) )}^{2} \qquad Equation 18$$

from the second horizontal (h), vertical (v), and diagonal (d) subbands of the stationary wavelet transform. r^{b}(x) is the coefficient at pixel x in subband b, squared and with scaling tuned to the image. μₖ^{b}(c) is mean energy at b for class c at k.

3. Analysis and Results

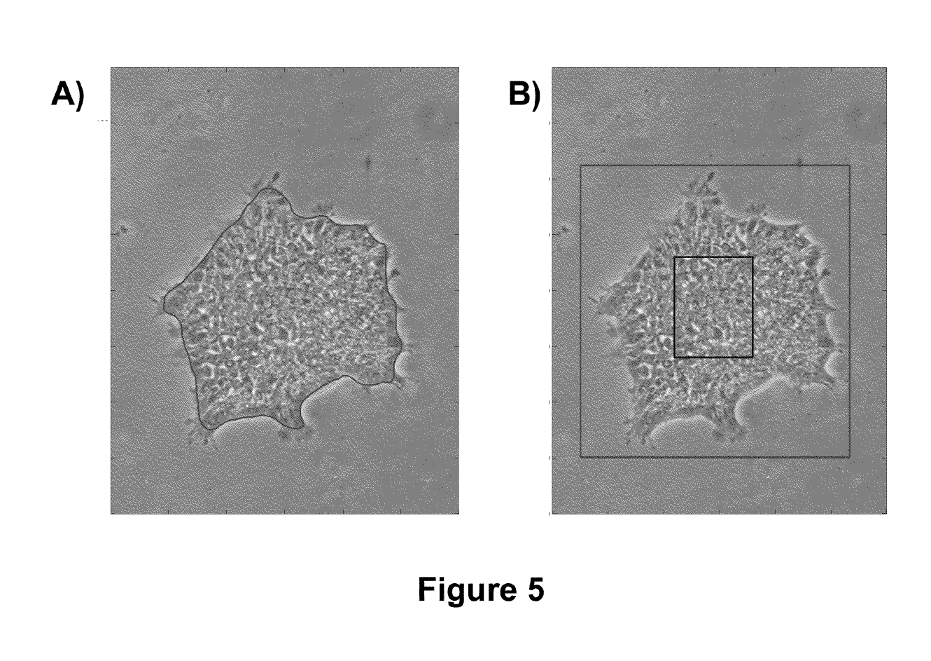

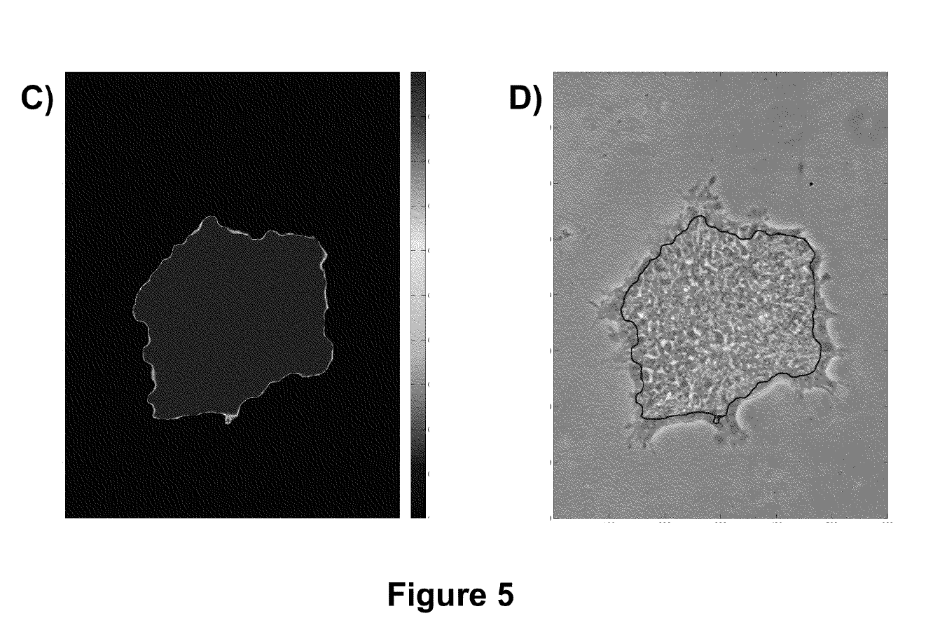

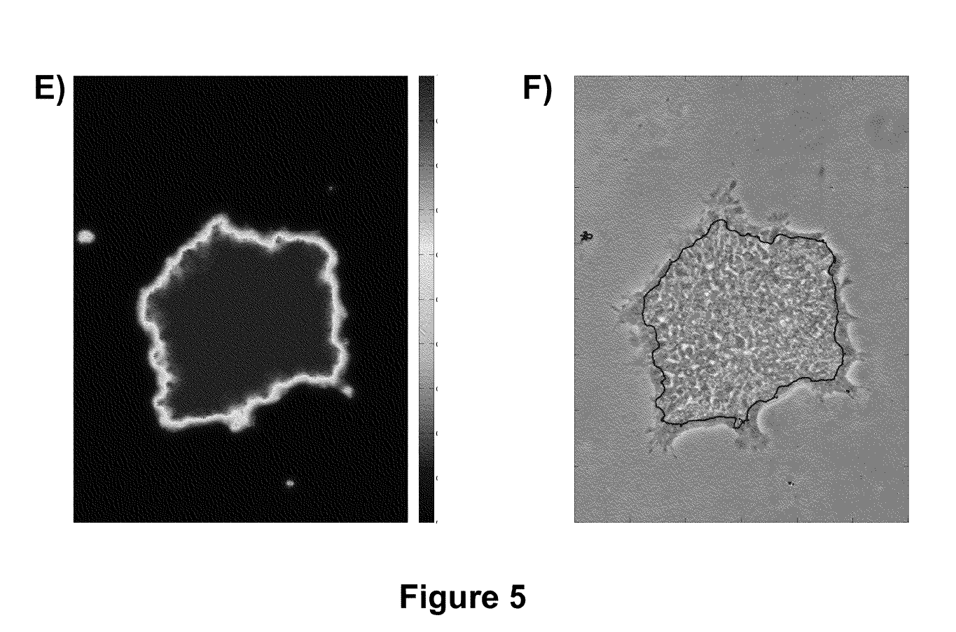

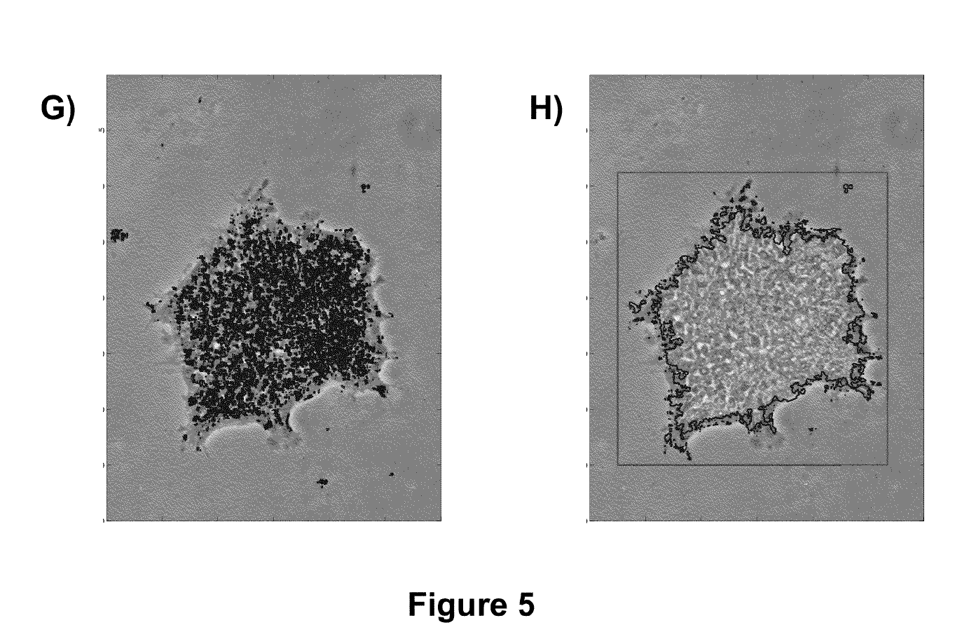

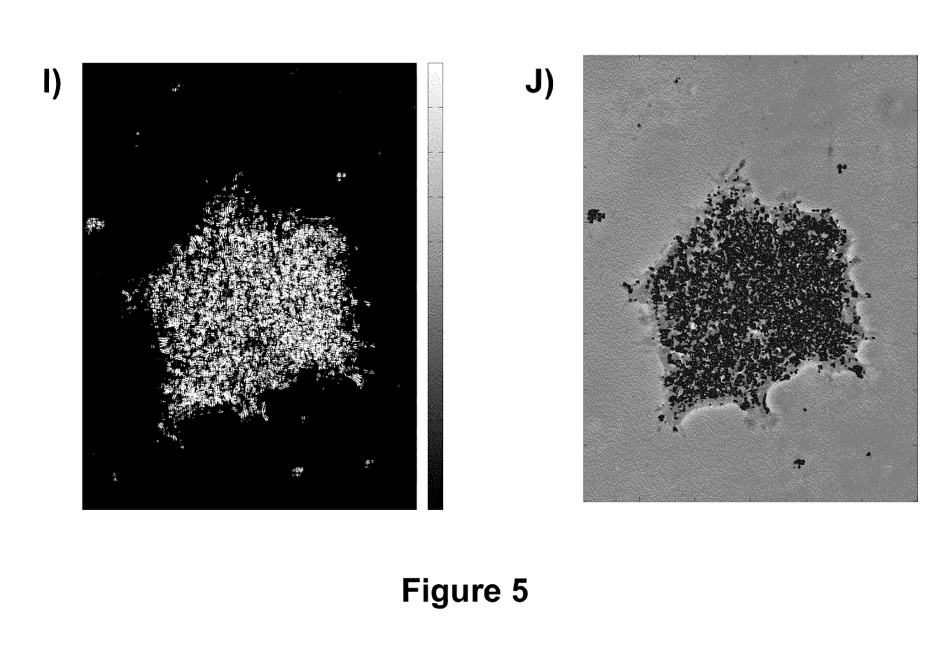

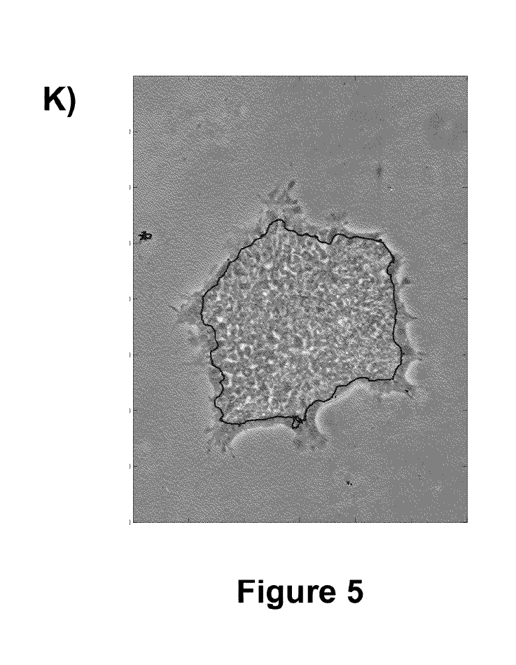

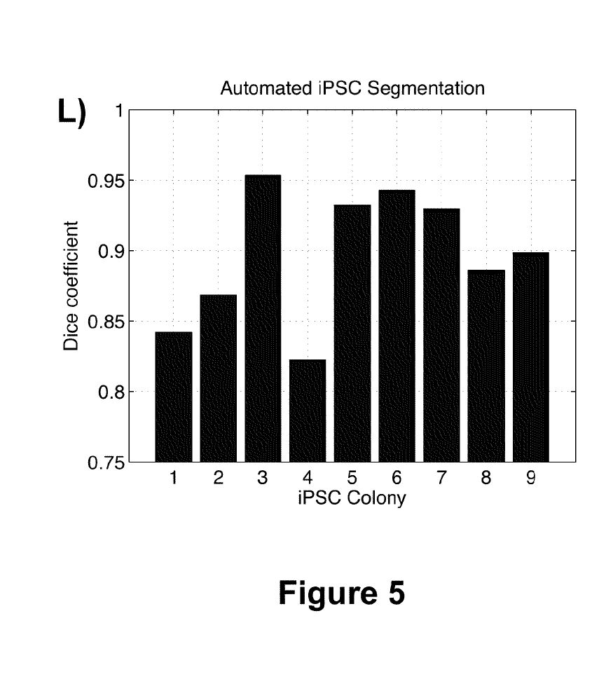

FIG. 5 shows the application of the disclosed unified algorithm to an iPSC colony. FIG. 5a shows the colony with ground truth established under direction of a microscopist outlined in red. Initial conditions are shown in FIG. 5b; the area within the blue contour is set to ρ₀(Z=c₀|Y)=1, between blue and red ρ₀(Z=c₀|Y)=½, and external to red ρ₀(Z=c₀|Y)=0. Results for the curvature (equation (10)) and Gaussian (equation (12), σ=4) priors are shown in FIGS. 5c and e, respectively. In both cases, v=½. In FIG. 5d, f, hard decision was recovered by thresholding at ρ₀(Z=c₀|Y)=½ in order to assess agreement with the ground truth via the Dice coefficient:

$$d = \frac{2 \vert A ⋂ B \vert}{\vert A \vert + \vert B \vert} \qquad Equation 19$$

A and B are sets identifying the image foreground. In FIG. 5, this corresponds to the area in which ρ(Z=|Y; θ)=½.

Both priors produced significant agreement with the ground truth; d=0.89, 0.90 for the curvature and Gaussian priors, respectively. Error was largely due to oversmoothing at borders. As flow-by-curvature and diffusion converge as ρ(Z|Y; θ)→0, 1 (B. Jawerth et al., J. Visual Communication and Image Representation. 13, 94-102 2002), the two solutions are nearly identical; the Dice coefficient between them is d=0.99. As the Gaussian prior is simpler to compute, it was used for automation.

In FIG. 5g-h, these results were compared to FMM-EM (prior as in (8), d=0.41) and level set (d=0.90) implementations. Due to the high variability of this feature set, adequate performance required enforcing local similarity, and the FMM-EM failed, finding only the feature peaks.

The algorithm was then used to automate the segmentation of a series of nine iPSC images. To automatically generate initial ρ₀(Z), each feature was independently thresholded layer and set ρ₀(Z=c₀)=1 if any one layer exceeded its threshold, as illustrated in FIG. 5i. FIGS. 5j-k compare level sets to the unified approach. Updating within the narrow band, the former found only the feature peaks, while the latter segmented correctly. FIG. 5I plotted Dice coefficients for the nine colonies segmented via the unified approach (mean d=0.90, standard deviation of 0.05).

### D. Application to Brain MRI

1. Methods

Magnetic Resonance Imaging (MRI), is widely used to noninvasively investigate brain structure and function. Given the high volume of image data generated, accurate, consistent, automated segmentation and analysis is required, but traditional methods can be susceptible to noise, which research such as Y. Zhang et al., IEEE Trans. Medical Imaging, Vol. 20, No. 1, January 2001, 45-57, seeks to mitigate.

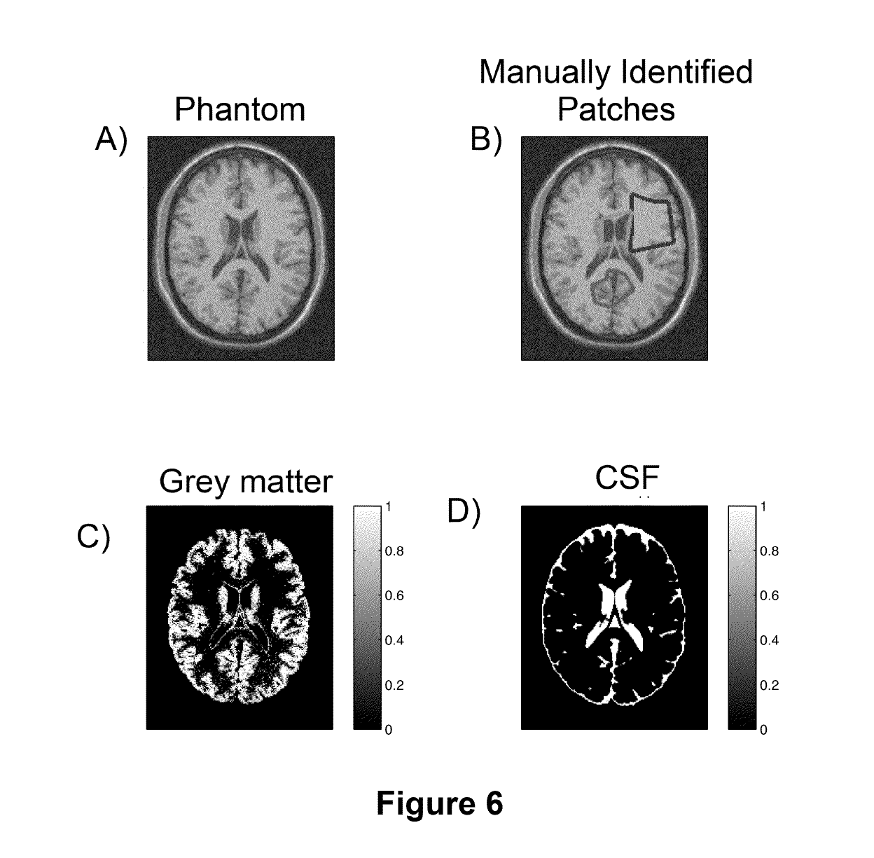

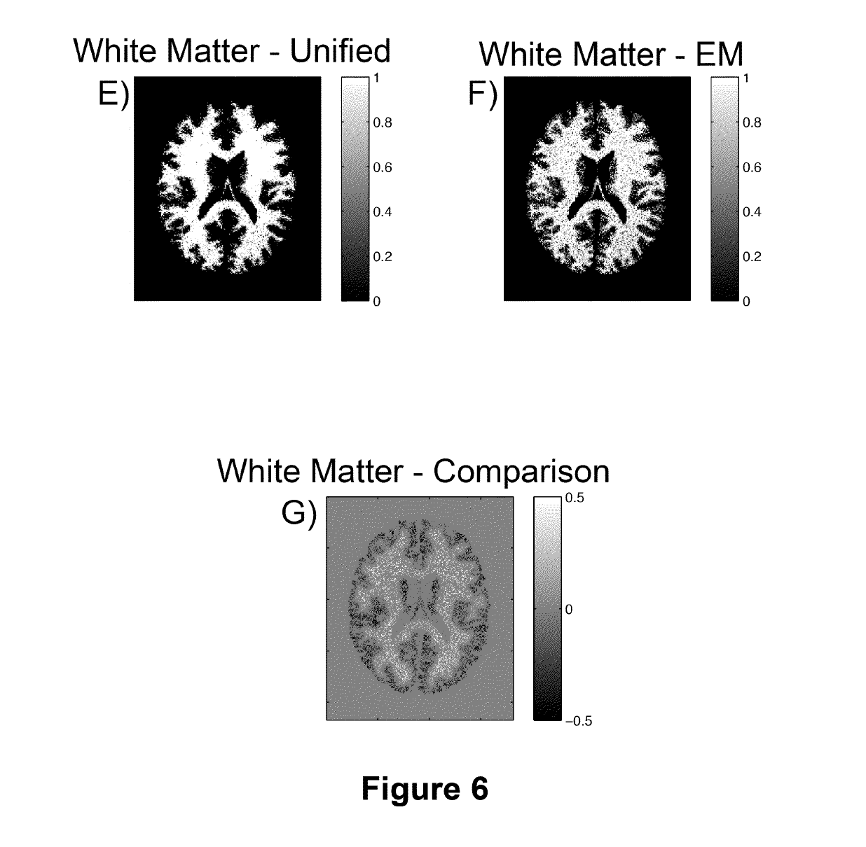

Demonstrated in this example is extensibility to multi-region segmentation and robustness to noise via application of the disclosed algorithm to brain MRI segmentation, a popular application of the FMM-EM. The Montreal Neurological Institute (MNI) brain phantom was used in this example (D. Collins et al., IEEE Trans. Medical Imaging. Vol. 17, No. 3, June 1998, 463-468) (FIG. 6a), corrupted by noise with 25% power.

2. Results

FIG. 6 shows a comparison of the disclosed unified approach to an FMM-EM in segmenting the phantom into white matter, gray matter, and cerebrospinal fluid (CSF). Both algorithms assume ρ(Y|Z; θ) to be Gaussian. The FMM-EM used mixture prior (8), while the unified algorithm used a Gaussian prior ((12), σ=3). Initial θ were determined from manually delineated patches (FIG. 6b; white matter is blue, gray matter red, and CSF green). No bias or gain correction was performed.

FIGS. 6c-e show results from the unified approach on gray matter, CSF, and white matter, respectively. In FIG. 6f, FMM-EM results are given for white matter. FIG. 6g compares the two algorithms by plotting FIG. 6e minus FIG. 6f. Lighter voxels indicate coordinates which the unified approach classifies more likely to be white matter and indicate its superior robustness to noise. Based on visual inspection of FIGS. 6e-g, the unified approach suppressed speckling, causing it to be more confident both in locating white matter in the correct region and rejecting it elsewhere.

As shown herein, a unified approach to EM and level set segmentation combines the advantages of both algorithms. This methodology retained the topological flexibility of level sets, but was simpler in implementation and more naturally extensible to multiple regions. Relative to the FMM-EM, this approach enhanced noise rejection and smoothed class transitions. The results demonstrated indicate that this approach is thus a significant step towards the automated, texture-based detection and analysis of iPSC colonies and promoted analysis and classification of highly degraded brain MRI images.

### Example 3

### Texton-Based Segmentation and Classification of Human Embryonic Stem Cell Colonies Using Multi-Stage Bayesian Level Sets

### A. Introduction

Disclosed herein is a texton-based, multi-stage Bayesian level set algorithm which is used to segment colony images of hESC and their derivatives. Previous research was extended to segmenting stem cells according to multiresolution texture methods to accommodate colonies and tissues with diffuse and varied textures via a filter bank approach similar to the MR8. Texture features computed for test images were classified via comparison with learned sets of class-specific textural primitives, known as textons. Encompassing this texture model is the new Bayesian level set algorithm, which smoothes and regularizes classification similar to level sets but is simpler in its probabilistic implementation. The resulting algorithm accurately and automatically classifies images of pluripotent hESC and trophectoderm colonies for high-content screening applications.

Pluripotent human embryonic stem cells (hESC) and their derivatives present exciting research opportunities but must generally be continually monitored to prevent unsupervised differentiation and insure successful fate control. Visual inspection offers a non-invasive alternative to destructive chemical testing, but sufficiently trained microscopists have limited time, are expensive to train, and may yield subjective results, acute shortcomings in high-throughput screening applications.

Despite enabling successful classification, wavelet texture methods exhibit some disadvantages in segmenting certain cell colony images, including trophectoderm (TE), a hESC derivative. Unlike the tight, textured hESCs, TE exhibits large, dark cells in a diffuse matrix and is better modeled by blob or bar detectors at particular scales rather than wavelet high-pass subbands.

Textons, which model texture according to a learned dictionary of feature vectors, require a multi-stage BLS that hierarchically proceeds from class (pluripotent, TE, growth media) through a library of textons to the image features. This novel approach automates the segmentation of pluripotent hESC and TE images, towards the long term goal of automatic stem cell cultivation and assessment of experiments.

### B. Methods

1. Texture-Based Bayesian Level Sets

a. Texton-Based Texture Classification

Recent texture classification efforts model texture via a set of primitives (i.e. textons) which define what a texture locally “looks like.” Several methods exist for calculating these textons, including statistical modeling of image patch exemplars (M. Varma, et al., IEEE Trans. PAMI 31.11 (November 2009) 2032-2047) and compressive sensing techniques (L. Liu, et al., “Texture Classification Using Compressed Sensing.” Canadian Conf. Compo and Robot. Vis. 2010), but we adopt the MR8 approach of M. Varma, et al., Int'l J. Computer Vision 62.112 (2005) 61-81, which models texture by convolving the image with a filter bank containing Gaussian (local mean) and Laplacian-of-Gaussian (LoG, blob detector) filters at fixed scale and edge and bar filters at three different scales and several orientations. The texton is the vector of eight filter responses at a given pixel.

To classify an image texture patch, a texton dictionary must first be learned for each class; μ_{t/c}, was denoted as the t^{th} learned feature vector for class c. Texturally homogeneous images may be classified by comparing the statistics of image textons to these learned dictionaries (M. Varma, et al. Int'l J. Computer Vision 62.112 (2005) 61-81). Texturally inhomogeneous images were segmented according to the multi-stage Bayesian level set algorithm described below.

b. Bayesian Level Sets

In N. Lowry, et al., ISBI 2011, the Bayesian level set algorithm was introduced to solve the level set problem in a manner similar to the finite mixture model Expectation-Maximization algorithm. Like level sets, the BLS rejects noise and produces segmentations with smooth, regular borders, but it is simpler in implementation, requiring no solution of PDEs or maintenance of the level set surface φ and is much more tolerant of poor initial conditions. Furthermore, the BLS may be easily extended to an arbitrary number of classes without requiring additional computational machinery to eliminate overlap between regions or fill gaps as in many multiphase level set algorithms.

Level set region competition (S. Zhu, et al., IEEE Trans. PAMI 18.9 (September 1996) 884-900) segments an image into classes c₀ and c₁ by minimizing a functional of surface φ:

$$\begin{matrix}E ( φ , Θ ; Y ) = λ {\int }_{Ω}^{ } ‖ ∇ H ( φ ( x ) ) ‖ d x \\ = - {\int }_{Ω}^{ } H ( φ ( x ) ) \cdot log p ( Y ( x ) \vert Z ( x ) = {c}_{0} ; Θ ) d x \\ = - {\int }_{Ω}^{ } H ( φ ( x ) ) \cdot log p ( Y ( x ) \vert Z ( x ) = {c}_{1} ; Θ ) d x\end{matrix} \qquad Equation 20$$

Z(x) is the true classification and assigns each pixel x in the image domain 12 label from set c. Observations Y(x) are generated stochastically from Z(x) according to parameters θ. The regularized Heaviside step function H(•) demarcates the two regions (c₀={x|θ(x)>0}), so the zero level set of φ partitions the image. Constant λ penalizes the length of the segmenting contour. The minimizing condition is:

$$\begin{matrix}0 = log \frac{p ( Y ( x ) \vert Z ( x ) = {c}_{0} ; Θ )}{p ( Y ( x ) \vert Z ( x ) = {c}_{1} ; Θ )} + λ k ( φ ( x ) ) & ∀ x ∈ Ω\end{matrix} \qquad Equation 21$$

where κ(φ) is the curvature of the iso-contours of φ.

By applying the “log-odds” approach of K. Pohl et al., IPMI 2007, LNCS 4584 (2007) 26-37:

$$φ ( x ) = log \frac{p ( Y ( x ) \vert Z ( x ) = {c}_{0} ; Θ )}{p ( Y ( x ) \vert Z ( x ) = {c}_{1} ; Θ )} \qquad Equation 22$$

a Bayesian procedure for solving for (21) is derived. The results are presented below, referring the reader to N. Lowry et al., ISBI 2011 for the derivation.

At each iteration k, the Bayesian Level Set (BLS) algorithm refines image classification in two steps. First, update soft classification ρ(Z|Y; θ) via Bayes' rule. Second, estimate parameters θ via maximum likelihood.

The second sub-iteration is self-explanatory, and we focus on the first. For compactness, we abbreviate as q_{Z|Y}^{k}(c) the probability at iteration k that pixel x is a member of class cεC conditioned on data Y(x) and parameters θestimated at the previous iteration, i.e. ρₖ(Z(x)=c|Y(x); θₖ₋₁). Likewise, prior and conditional probabilities ρₖ (Z(X)=c) and ρₖ (Y(x)|(Z(x)=c); θₖ₋₁) are q_{Z}^{k}(c) and q_{Y|Z}^{k}(c). Update according to Bayes' rule:

$${q}_{Z \vert Y}^{k} ( c ) = \frac{{q}_{Y \vert Z}^{k} ( c ) {q}_{Z}^{k} ( c )}{{\sum }_{k ∈ c}^{ } {q}_{Y \vert Z}^{k} ( l ) {q}_{Z}^{k} ( l )} \qquad Equation 23$$

The smoothing prior q_{Z}^{k+1} is calculated from the previous log-likelihood of classification, smoothed via flow-by-curvature:

$$\begin{matrix}{q}_{Z}^{k} ( c ) = {( 1 + {e}^{- {k}^{k - 1 ( c )}} )}^{- 1} \\ {L}^{k - 1} ( c ) = log \frac{ {q}_{Z \vert Y}^{k - 1} ( e )}{1 - {q}_{X \vert Y}^{k - q} ( c )} + λ k ( {q}_{Z \vert Y}^{k - 1} ( c ) )\end{matrix} \qquad Equation 24$$

Multi-region smoothing priors are then normalized to one.

With this smoothing prior, (23) reaches steady state at the minimizing condition in (21). Such a prior effectively creates stochastic dependence in a localized neighborhood in order to encourage local classification similarity and is analogous to atlas-based EM classification. In the absence of a pre-defined probabilistic classification atlas, one by smoothing was iteratively estimated for the previous soft classification. In certain applications, it may be desirable to substitute other smoothing functions for flow-by-curvature, e.g. Gaussian diffusion.

c. Multi-stage Bayesian Level Sets

The BLS implicitly assumes that observations Y are generated via a two-stage Markov process: First, a class is selected for each pixel according to some prior function q_{z}, after which observations are generated from parameters θ.

Texton-based classification is accommodated by adding an intermediate stage to the BLS. As before, class Z was determined via a prior function. Next, a texton T is selected from a set of 20 primitives T_{z} specific to class Z according to a second prior function ρ(T|Z; θ), denoted q_{T\Z}. Lastly, Y was generated from T based p(Y|T; θ), denoted q_{T\Z}, which is conditionally independent of Z. The intuition is that the class should be locally similar, but textons within a class may be arbitrarily jumbled; thus e priors were defined that admit dependence on Z but allow for spatial independence of the textons within Z.

At iteration k, sub-iteration 1, q_{Z|Y}^{k}(c) is determined by marginalizing with respect to T_{c}:

$${q}_{Z \vert Y}^{k + 1} ( c ) = \frac{{q}_{Z}^{k + 1} ( c ) {\sum }_{t ∈ {τ}_{c}}^{ } {q}_{T \vert Z}^{k + 1} ( t , c ) {q}_{Y \vert T}^{k + 1} ( t )}{{\sum }_{l ∈ c}^{ } {q}_{Z}^{k + 1} ( l ) {\sum }_{s ∈ {τ}_{l}}^{ } {q}_{T \vert Z}^{k + 1} ( s , l ) {q}_{Y \vert T}^{k + 1} ( s )} \qquad Equation 25$$

q_{T|Z}^{k}(t,c) is a constant π_{t|c}^{k}, and q_{Y|T}^{k}(t) is normal about μ_{t|c} with covariance Σ_{t|c}^{k}:

$$\begin{matrix}{q}_{T \vert Z}^{k} ( t , c ) = {π}_{t \vert c}^{k} & Equation 26 \\ {q}_{T \vert Z}^{k} ( t , c ) \sim N ( Y ; {μ}_{t \vert c} {\sum }_{t \vert c}^{ k} ) & Equation 27\end{matrix}$$

In sub-iteration 2, parameters are estimated:

$$\begin{matrix}{π}_{t \vert c}^{k + 1} = \frac{1}{N ( c )} {\sum }_{Ω }^{ } {q}_{Z \vert Y}^{b + 1} ( c ) {q}_{T \vert Y , Z}^{k + 1} ( t , c ) & Equation 28 \\ {\sum }_{t \vert c}^{k + 1} = {\sum }_{Ω} [ {q}_{Z \vert Y}^{k + 1} ( c ) {q}_{T \vert Y , Z}^{k + 1} ( t , c ) - ( Y - {μ}_{t \vert c} ) {( Y - {μ}_{t \vert c} )}^{T} ] & Equation 29 \\ N ( c ) = {\sum }_{Ω}^{ } {q}_{Z \vert Y}^{k + 1} ( c ) {\sum }_{s ∈ {T}_{c}}^{ } {q}_{T \vert Y , Z}^{k + 1} ( s , c ) & Equation 30 \\ {q}_{T \vert Y , Z}^{k + 1} ( t , c ) = \frac{{q}_{Z \vert Y}^{k + 1} ( c ) {q}_{T \vert Y , Z}^{k + 1} ( t , c )}{{\sum }_{s ∈ {T}_{c}}^{ } {q}_{Y \vert T}^{k + 1} ( s ) {q}_{T \vert Z}^{k + 1} ( s , c )} & Equation 31\end{matrix}$$

μ_{t|c} are constant textons learned a priori.

### C. Application to Stem Cell Colonies

1. Data Collection

In some implementations, pluripotent hESCs, line H7, were grown feeder-free on Geltrex-coated plates (Invitrogen) and maintained in StemPro (Invitrogen), a defined pluripotency media. Media was changed every other day and colonies were passaged weekly with Collagenase Type IV. Specific and selective differentiation to an epithelial cell type was achieved by culturing for four days in EMIM basal media without bFGF but with the addition of BMP-4 (100 ng/mL) to the media (Erb, et al., Stem Cells Dev. 20.9, September 2011).

In some implementations, colonies were imaged via phase-contrast microscopy using a 4× Nikon 0.13 NA objective on a TMS cell culture microscope with a 10M Pixel Nikon D40× SLR camera. In some of these implementations, these images are used directly, with the pixels analyzed is subsequent steps being the original pixels captured. In other implementations, the image captured is altered before the analysis steps begins. For example, the original image may be cropped or the resolution reduced such that the analyzed image is 1024×1024 pixels in size. In other implementations, neighboring pixels are combined with an imaging filter. In some implementations there is no constraint to the number of pixels or dimension of the captured image.

In some implementations, the textons are classified using the above descried Bayesian Level Sets. In some implementations, each pixel is classified as belonging to a cell colony, a cell colony border, or the growth medium. In some implementations, the interior of the defined colonies are analyzed to identify regions devoid of cells. In some implementations, the regions devoid of cells includes growth medium where no cells are actively growing. In other implementations, the regions devoid of cells are areas of the cell colony contained dead cells.

2. Texture Feature Selection and Training

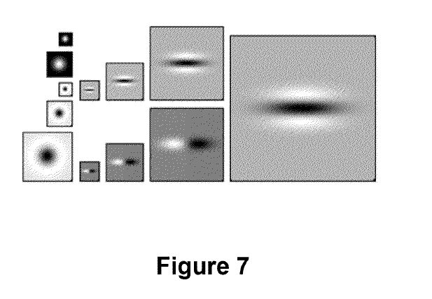

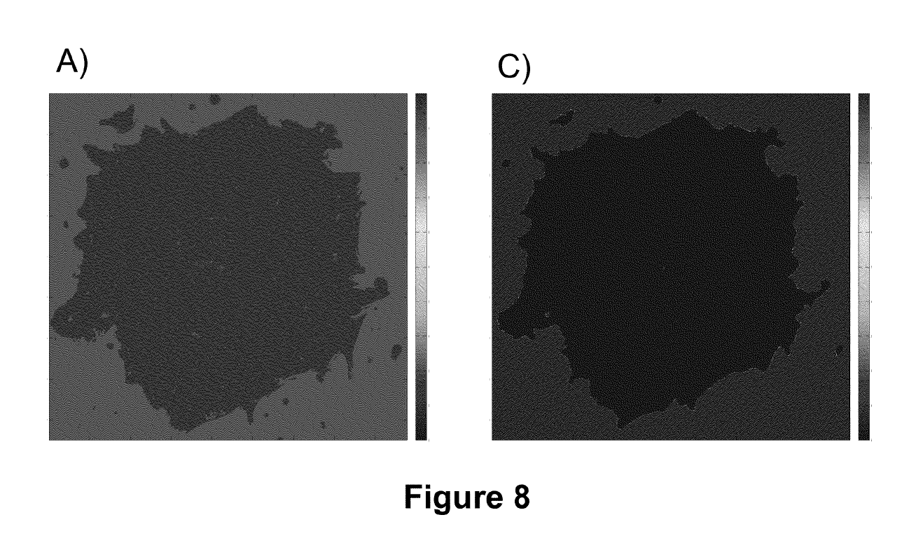

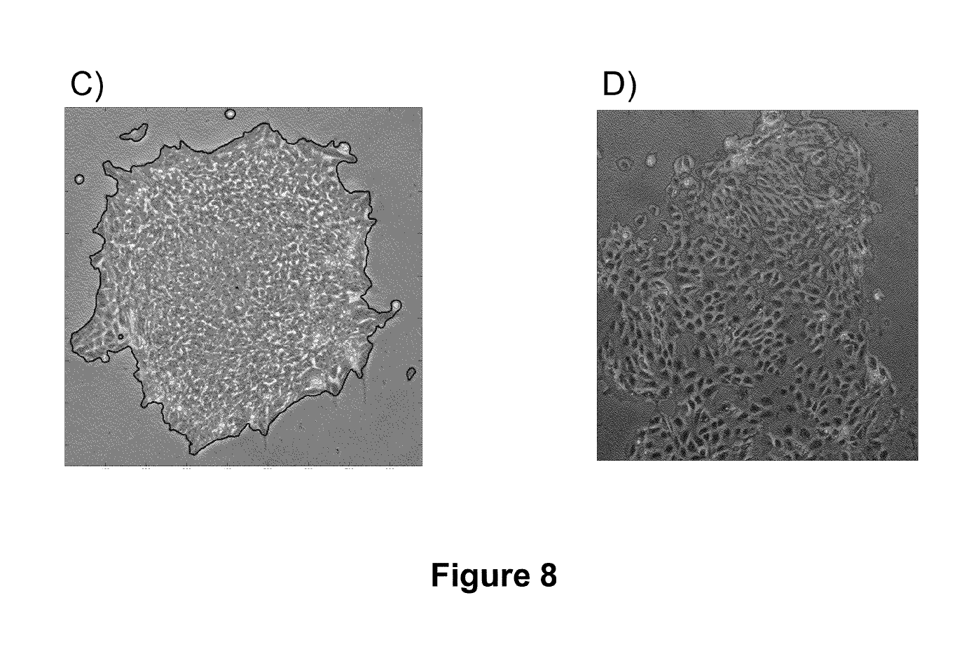

In some implementations, the eight MR8 features were scaled to a particular database (M. Varma, et al., Int'l J. Computer Vision 62.112 (2005) 61-81) so eighteen feature were examined to select the ones most relevant to data of interest. Specifically, Gaussian and LoG at σ={2, 4, 8, 16, 32} and bar and edge filters at σ={1, 2, 4, 8}. By manually labeling images in FIG. 8c-d as pluripotent, TE, and growth medium, twelve most relevant features were selected according to mutual information (MI): Gaussian at σ={2, 4}, LoG at σ={2, 4, 8}, bar filters at σ={1, 2, 4, 8}, and edge filters at σ={1, 2, 4}. Selected filters are shown in FIG. 7.

Using a greedy algorithm, a texton library was chosen for the three classes that maximized the number of elements in the training set which were closer (according to Mahalanobis distance) to a corresponding library element than to any texton in the training set with different class label.

3. Results and Analysis

In some implementations, the textons are classified using the multi-stage BLS described above. FIG. 8 illustrates the training and segmentation process for the texton-based, multi-stage BLS applied to pluripotent hESC and TE cell colonies. In FIG. 8a, initial conditions were determined by finding the texton with the minimum Euclidean distance to a particular pixel and setting the corresponding class probability to 0.9. Final probabilities are in FIG. 8b, and contours in FIG. 8c indicate where q_{Z|Y}=0.5. Regions interior to the contour are classified as cell colony and exterior regions as growth media.

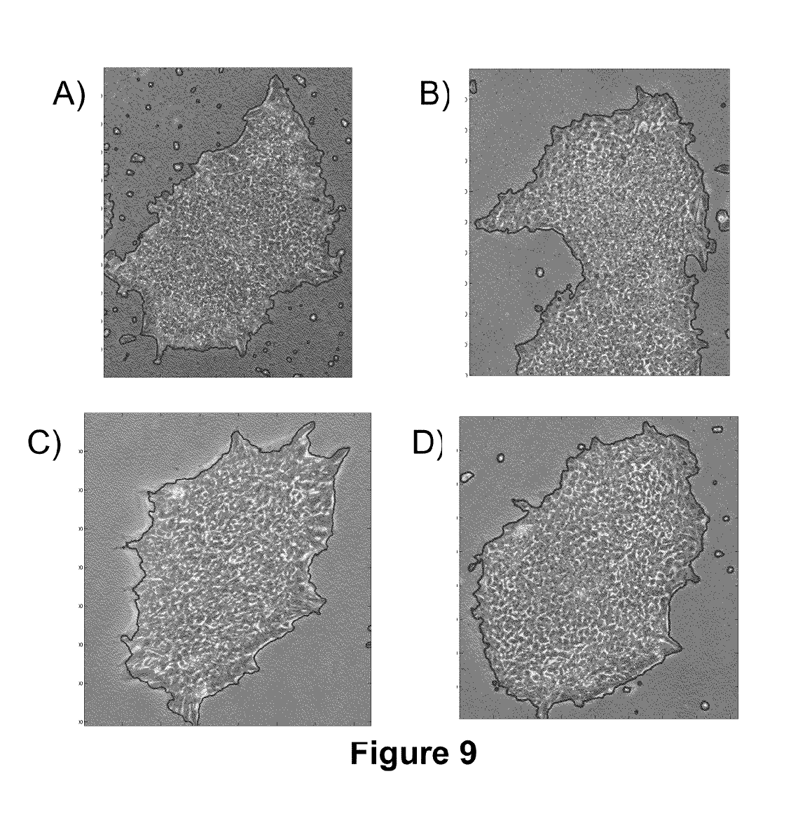

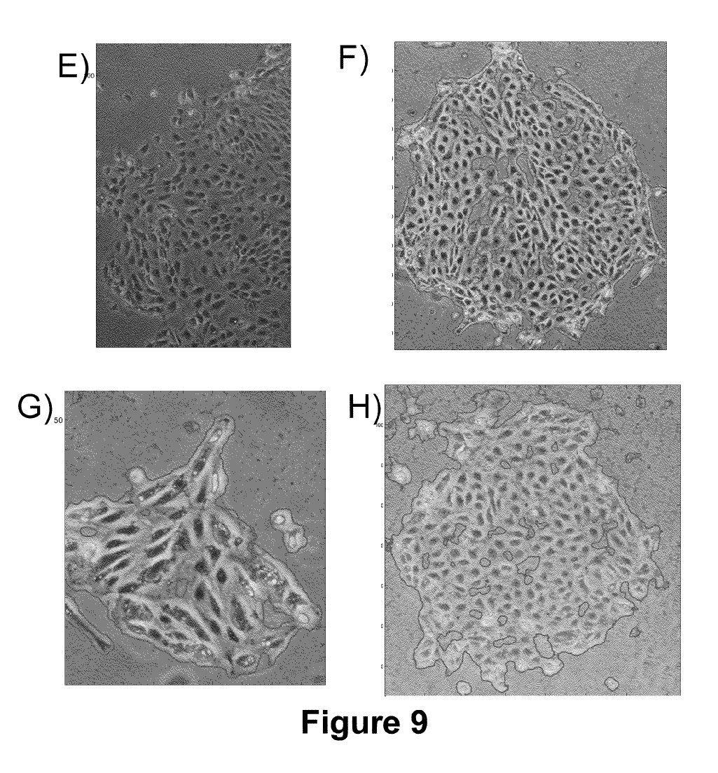

FIG. 8c shows the segmentation of the image used to train the pluripotent texton library. The colony is correctly segmented, with a tight border excluding most of the phase halo—an imaging artifact manifesting as a white band around the colony. This artifact has a significant filter response, so simple thresholding in the texture space would assign it to the colony; in some implementations, the texture library should be trained to account for this. False positives included dead cells (white blips at the top and top left) and extracellular protein (black streak at right). With similar scale and appearance to the colony, these are difficult to distinguish texturally. Pluripotent test images segmented according to this library exhibit similar performance and are shown in FIGS. 9a-d.

FIG. 8d shows the segmentation of the image used to train the TE library. These colonies are fairly diffuse, yet the algorithm successfully found internal holes that are too tedious for manual delineation. Test images classified based on this library are shown in FIGS. ge-h and exhibit similar performance. FIG. 9h is of interest; despite the perceptible visual difference between the colony and the growth media in the upper right, their wavelet subband coefficients are statistically similar, making these regions difficult to distinguish using multiresolution methods. Save for a few misclassified regions, the texton-based, multi-stage BLS accurately locates the colony border.

Multi-stage Bayesian level sets allow for texture-based segmentation using learned texton dictionaries. Though the disclosed approach uses filter bank methods; this scheme is easily extensible to texton models such as patch exemplars or compressive sensing. Like level sets, the BLS encompassing the texture model encourages smoothness in the segmentation to regularize classification and suppress noise but is simpler in implementation. Results shown indicate present ability to successfully and automatically segment pluripotent hESC and trophectoderm colonies despite the presence of distracting external objects, including dead cells and extracellular protein. Such technology can be extended to the texton library to additional colony and tissue types.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific implementations of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed:

1. A method of identifying borders of a cluster of neighboring cells, comprising:
obtaining an image of a cluster of neighboring cells;
representing the image as a multiplicity of pixels;
segmenting the image using texton analysis, thereby identifying the borders of the cluster of neighboring cells; and
identifying segments devoid of cells between at least some cells within the cluster of neighboring cells.

2. The method of claim 1, which does not require a user to manually identify the cluster of neighboring cells.

3. The method of claim 1, wherein calculating a texton comprises calculating at least eight filter responses at a given pixel.

4. The method of claim 3, wherein at least one of the filter responses is derived from a Gaussian filter, a Laplacian-of-Gaussian filter, or a bar filter.

5. The method of claim 1, wherein the cluster of neighboring cells is a stem cell colony, a colony of differentiated cells, a colony of trophectoderm cells, or a colony of neuronal cells.

6. The method of claim 1, wherein the texton analysis comprise analyzing cell texture.

7. The method of claim 6, wherein analyzing cell texture comprises performing wavelet decomposition analysis or any multiresolution decomposition algorithm.

8. The method of claim 7, wherein the wavelet decomposition analysis or multiresolution decomposition algorithm is an n-level decomposition that yields three detail subbands per level.

9. A method of classifying test cells, comprising:
obtaining an image of one or more test cells;
representing the image as a multiplicity of pixels;
using a processor to calculate a texton of at least a subset of said multiplicity of pixels, wherein calculating the texton comprises calculating at least one filter response at a given pixel;
using a processor to compare the texton to one or more reference textons using one or more statistical comparison methods; and
identifying the reference cell that most closely matches the test cell based on the comparison; whereby the test cells are classified as belonging to a class corresponding to the identified reference.

10. The method of claim 9, wherein calculating the texton comprises calculating at least eight filter responses at a given pixel.

11. The method of claim 9, wherein calculating a texton comprises using a Gaussian filter, a Laplacian-of-Gaussian filter, or a bar filter.

12. The method of claim 9, wherein the texton analysis comprise analyzing cell texture.

13. The method of claim 9, wherein analyzing cell texture comprises performing wavelet decomposition analysis or any multiresolution decomposition algorithm.

14. The method of claim 9, wherein the wavelet decomposition analysis or multiresolution decomposition algorithm is an n-level decomposition that yields three detail subbands per level.

15. The method of claim 9, which is performed on cells within a border identified using a segmentation algorithm.

16. A method of classifying test cells, comprising:
obtaining an image of one or more test cells;
representing the image as a multiplicity of pixels;
analyzing, by a processor, a texture of at least a subset of said multiplicity of pixels;
comparing the texture with at least five library textures derived from a library of reference cells, wherein the processor applies one or more statistical comparison methods to compare the textures, and wherein the library comprises cells of at least three differentiation states; and
identifying the reference cell that most closely matches the test cell based on the comparison, whereby the test cells are classified as belonging to a class corresponding to the identified reference cell.

17. The method of claim 16, wherein the library further comprises at least two different lineages.

18. The method of claim 16, wherein the reference cell types in the library are selected from at least two of a mouse cell, a human cell, an embryonic stem cell, an induced pluripotent cell, a neural stem cell, a kidney cell, a trophectoderm cell, a neurectoderm cell, a fibroblast, and an oligodendrocyte precursor cell.

19. The method of claim 16, wherein the one or more statistical comparison methods comprise a comparison of probability density functions, or estimates thereof.

20. The method of claim 16, wherein the one or more statistical comparison methods comprise a Kullback-Leibler Distances comparison.

21. The method of claim 16, wherein the one or more statistical methods comprise a parametric or non-parametric binary or M-ary hypothesis test.

22. The method of claim 16, wherein analyzing cell texture comprises performing wavelet decomposition analysis or any multiresolution decomposition algorithm.

23. The method of claim 22, wherein the wavelet decomposition analysis or multiresolution decomposition algorithm is an n-level decomposition that yields three detail subbands per level.

24. A method of comparing cells cultured by different users, comprising:
providing a database suitable for storing cell culture condition data and cell image data;
receiving cell culture condition data and cell image data provided by a first user;
calculating, by a processor, a similarity between the cell image data and cell culture condition data provided by the first user to the cell image data and cell culture condition data previously stored in the database using one or more statistical comparison methods; and
transmitting the similarity to at least the first user.

25. The method of claim 24, further comprising;
receiving cell culture condition data and cell image data provided by a second user; and
calculating a second similarity between the cell image data and cell culture condition data provided by the first user to the cell image data and cell culture condition data provided by the second user using one or more statistical comparison methods.

26. The method of claim 24, further comprising;
receiving cell culture condition data and cell image data provided by a third user; and
calculating a third similarity between the cell image data and cell culture condition data provided by at least two of the first user, the second user, and the third user using one or more statistical comparison methods.

27. The method of claim 25, wherein the first use is different than the second user.

28. The method of claim 24, wherein the cell image data comprises a micrograph, textural information derived from a micrograph, or information derived from a micrograph using wavelet decomposition.

29. The method of claim 25, wherein the micrograph is obtained by phase contrast microscopy, fluorescence microscopy, or electron microscopy phase contrast, fluorescence microscopy, luminescence microscopy, magnetic resonance imaging, ultrasound imaging, or widefield microscopy, confocal microscopy, tomographic reconstruction, or statistical reassignment.

30. The method of claim 24, further comprising displaying the cell culture condition data and cell image data previously stored in the database to the first user.

31. The method of claim 24, wherein the cell culture condition data provided by the first user or the second user comprises conditions appropriate for long-term cell maintenance or experimental conditions.

32. The method of claim 24, wherein the first user accesses the database via the internet.

33. The method of claim 24, wherein the one or more statistical comparison methods comprise a comparison of probability density functions, or estimates thereof.

34. The method of claim 24, wherein the one or more statistical methods comprise a parametric or non-parametric binary or M-ary hypothesis test.

35. The method of claim 24, wherein the similarity is calculated using a Probability Density Function estimator and quantified using information divergence.

36. The method of claim 24, wherein calculating the similarity comprises applying Kullback-Leibler Distances.

37. The method of claim 24, wherein a processor identifies similarity between image data of a first cell and image data of a second cell, and predicts non-image data about the first cell based on non-image data about the second cell.

38. The method of claim 24, wherein the non-image data comprises gene expression data, protein level data, small molecule level data, or enzymatic activity data.

39. A system that compares cells, comprising:
a storage device that stores:
cell culture condition data provided by a first user and cell image data provided by the first user; and
cell culture condition data provided by a second user and cell image data provided by the second user; and
a computer application configured to:
calculate a similarity between the cell image data and cell condition data provided by the first user to the cell image data provided by the second user using one or more statistical comparison methods; and
transmit the similarity to at least one of the first user and the second user.

40. The system of claim 39, wherein the storage device further stores cell culture condition data provided by a first user and cell image data provided by the first user, and the computer application is further configured to calculate the similarity between the cell image data and cell culture condition data provided by at least two of the first user, the second user and the third user.

41. The system of claim 39, wherein cell image data comprises at least one of a micrograph, textual information derived from a micrograph, and information derived from a micrograph using wavelet decomposition.

42. The system of claim 39, wherein the first user is a different user than the second user.

43. The system of claim 40, wherein the micrograph is obtained by phase contrast microscopy, fluorescence microscopy, or electron microscopy.

44. The system of claim 39, wherein the cell culture condition data provided by the first user or the second user comprises conditions appropriate for long-tem cell maintenance or experimental conditions.

45. The system of claim 39, wherein the first and second user are connected to the storage device by a network.

46. The system of claim 39, wherein the one or more statistical comparison methods comprises a comparison of probability density functions, or estimates thereof.

47. The system of claim 39, wherein the one or more statistical comparison methods comprises a parametric or non-parametric binary or M-ary hypothesis test.

48. A bioinformatic method for predicting a characteristic of a test cell, comprising:
providing an electronic library comprising non-invasively obtained image data derived from reference cells, wherein the reference cells represent at least two differentiation states and at least two different lineages, wherein the electronic library further comprises molecular data gathered from the reference cells;
receiving a non-invasively obtained image of the test cell;
representing the non-invasively obtained image of the test cell as a multiplicity of pixels;
deriving image data from the multiplicity of pixels;
comparing, by a processor, the image data to non-invasive image data of the electronic library, wherein the processor applies one or more statistical comparison methods to compare the image data;
identifying a reference cell that most closely matches the test cell based on the comparison; and
predicting that the test cell has a characteristic similar to a characteristic of the identified reference cell, wherein the reference cell characteristic is derived from the molecular data stored in the electronic library in relation to the identified reference cell.

49. The bioinformatic method of claim 48, wherein the non-invasive image data is a light micrograph of a living cell.

50. The bioinformatic method of claim 48, wherein the molecular data is non-image data.

51. The bioinformatic method of claim 48, wherein the molecular data is characteristic of a cell identity, disease state, or lineage type.

52. The bioinformatic method of claim 48, wherein the molecular data comprises immunofluorescence data, gene expression data, mRNA and miRNA level data, protein level data, small molecule level data, or enzymatic activity data.

53. The bioinformatic method of claim 48, further comprising verifying whether the test cell has the predicted characteristic.

54. A bioinformatic method for comparing a test cell to an electronic library of reference cells, comprising:
providing an electronic library comprising non-invasively obtained image data derived from reference cells, wherein the reference cells represent at least two differentiation states and at least two different lineages, wherein the electronic library further comprises molecular data gathered from the reference cells;
receiving molecular data gathered from the test cell;
receiving a non-invasively obtained image of the test cell;
representing the non-invasively obtained image of the test cell as a multiplicity of pixels;
deriving image data from the multiplicity of pixels;
comparing, by a processor, the image data derived from the multiplicity of pixels to non-invasive image data of the electronic library, wherein the processor applies one or more statistical comparison methods to compare the image data;
comparing, by a processor, the molecular data gathered from the test cell with molecular data gathered from the reference cells; and
identifying a reference cell that most closely matches the test cell based on the comparisons of image data derived from the multiplicity of pixels to the non-invasive image data of the electronic library and the molecular data gathered from the test cell to the molecular data gathered from the reference cells.

55. The bioinformatic method of claim 54, wherein the non-invasive image data is a light micrograph of a living cell.

56. The bioinformatic method of claim 54, wherein the molecular data is non-image data.

57. The bioinformatic method of claim 54, wherein the molecular data comprises immunofluorescence data, gene expression data, protein level data, small molecule level data, or enzymatic activity data.

58. A method of identifying borders of a cluster of neighboring cells, comprising:
obtaining an image of a cluster of neighboring cells;
representing the image as a multiplicity of pixels; and
segmenting the image using a unified expectation-maximization and level set analysis, thereby identifying the borders of the cluster of neighboring cells.

59. The method of claim 58, wherein the cluster of neighboring cells is a stem cell colony, a colony of differentiated cells, or brain tissue.

60. The method of claim 58, wherein the expectation maximization and level set analysis comprises analyzing cell texture.

61. The method of claim 60, wherein analyzing cell texture comprises performing wavelet decomposition analysis or any multiresolution decomposition algorithm.

62. The method of claim 61, wherein the wavelet decomposition analysis or multiresolution decomposition algorithm is an n-level decomposition that yields three detail subbands per level.
